SEQUENCE LISTING (6 sequences)

SEQ ID NO: 1 - protein, 14 aa, Artificial Sequence
  Synthetic polypeptide
KKKKKKKKKKKKKK

SEQ ID NO: 2 - protein, 15 aa, Artificial Sequence
  Synthetic polypeptide
KKKKKKKKKKKKKKC

SEQ ID NO: 3 - protein, 15 aa, Artificial Sequence
  Synthetic polypeptide
CKKKKKKKKKKKKKK

SEQ ID NO: 4 - protein, 20 aa, Artificial Sequence
  Synthetic polypeptide
KKKKKKKKKKKKKKKKKKKK

SEQ ID NO: 5 - protein, 8 aa, Artificial Sequence
  Synthetic polypeptide
CALCEFLG

SEQ ID NO: 6 - protein, 13 aa, Artificial Sequence
  Synthetic polypeptide
SECVEVFQNSCDW

(19) United States
(12) Patent Application Publication
(10) Pub. No.: US 2017/0097353 A1
(43) Pub. Date: Apr. 6, 2017

(54) Polymer Viral Compositions

(71) Applicant: The Regents of the University of California, Oakland, CA (US)

(72) Inventors: Gregory A. Weiss, Irvine, CA (US); Kritika Mohan, Irvine, CA (US)

(21) Appl. No.: 15/287,489

(22) Filed: Oct. 6, 2016

Related U.S. Application Data
(60) Provisional application No. 62/237,781, filed on Oct. 6, 2015.

(51) Int. Cl. G01N 33/574; A61K 35/768

(52) U.S. Cl. CPC G01N 33/57434; A61K 35/768

(57) ABSTRACT

There are provided, inter alia, viral compositions including a viral particle in contact with a polymer and the polymer is linked to a recognition moiety, and methods of use thereof.

### CROSS-REFERENCES TO RELATED APPLICATIONS

This application claims the benefit of U.S. Provisional Application No. 62/237,781, filed Oct. 6, 2015, the content of which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with Government support under grant numbers 1 R43 AI074163 and 1 R43 CA050779, awarded by The National Institutes of Health. The Government has certain rights in this invention.

### INCORPORATION BY REFERENCE OF SEQUENCE LISTING

The contents of the text file named 48538-524001US_ST25.TXT, which was created on Sep. 25, 2016, and is 3,307 bytes in size, are hereby incorporated by reference in their entireties.

### BACKGROUND

M13 bacteriophage is type of virus that infects only bacteria, and can be genetically modified to present ligands on its surface. Phage have also been incorporated into nanomedicine platforms for targeted drug delivery and imaging. Such applications require low background binding by phage to cell surfaces. Phage typically adheres to cell surfaces with high affinity. Such non-specific adhesion complicates the design of phage-based sensors for the detection of tumor cells.

There are provided herein solutions to these and others problems in the art.

### SUMMARY

In one aspect, provided herein is a virial composition. The virial composition includes (a) a whole viral particle comprising a charged protein coat that has a plurality of charged coat proteins; (b) a first polymer electrostatically bound to the plurality of charged coat proteins; and (c) a covalent linker linking the first polymer to a recognition moiety.

In another aspect, there is provided a complex that includes any virial composition described herein and a cell, where the recognition moiety of the virial composition is bound to the cell.

In another aspect, there is provided a pharmaceutical composition that includes any virial composition described herein and a pharmaceutically acceptable carrier, diluent or excipient.

In another aspect, there is provided a method for detecting a cancer cell in a subject. The method includes (a) contacting a biological sample of the subject with one or more virial compositions described herein, where the recognition moiety of one or more virial compositions is a cell surface marker binding moiety, and (b) detecting a cell-virial composition complex, and presence of the complex indicates presence of a cancer cell in the subject.

### BRIEF DESCRIPTION OF THE DRAWINGS

FIG. 1. Schematic illustration of the on-phage cycloaddition reaction to bioconjugate PEG polymers to the phage surface. Phage are first wrapped with K₁₄-alkyne, and then conjugated to different lengths of azide-functionalized PEG polymers. Sequence legend: K₁₄-alkyne (SEQ ID NO:1).

FIG. 2. Bar graph depicting phage-based ELISA demonstrating the effectiveness of wrapping phage by click chemistry with the indicated PEG azides to reduce non-specific adhesion to cellular surfaces. A >75% reduction in non-specific adhesion to LNCaP cells is observed for PEG45 compared to unwrapped phage. A lower HRP signal indicates decreased non-specific adhesion. Throughout this report, LNCaP cells are targeted at 4.5×10⁶ cells/mL, and error bars for ELISA data represent standard error (n=3). All experimental data points include such error bars, though often these are quite small. The p-value is <0.01 for all data reported here. Histogram bins (in order left to right): No wrap K₁₄-alkyne, PEG7, PEG22, PEG45, PEG100, no cells.

FIGS. 3A-3B. Bar graphs depicting dynamic light scattering measurements which indicate the consistent increase in size with the addition of wrappers on phage. Labels indicate the measured average size in nm for each indicated phage. Histogram bins (in order left to right): (FIG. 3A) PEG100 (no phage), no wrap, K₁₄-Cys (SEQ ID NO:2), PEG100; (FIG. 3B) P100_{SP}-P4-2 (all with phage). Y-axis for FIG. 3B is as disclosed for FIG. 3A.

FIG. 4. Bar graph depicting phage-based ELISA demonstrating the effectiveness of the bidentate binding mode for the two PSMA binding ligands on the phage surface. A higher HRP activity indicates stronger binding affinity between the displayed ligands and PSMA on the cell surface. The various ratios of wrapped ligands (red bars) can be compared with wrapping by individual ligands (patterned red bars). A 1:1 ratio of the two ligands indicates the assay of equimolar amounts of each ligand. Negative controls (gold bars) were as previously described. Histogram bins (in order left to right): no cells, PEG45, ratios of P100_{NSP}-2:P100_{NSP}-1 of 1:0, 3:1, 2:1, 1:1, 1:2 and 0:1.

FIGS. 5A-5B. (FIG. 5A) Phage-based ELISA comparing the different attachment modes with the incorporation of a PEG4 linker for PEGylated ligand 2 targeting PSMA on LNCaP cells. Patterned bars indicate non-specific (NSP) attachment modes. Histogram bins (in order left to right): No cells, PEG45, P100-2 with NSP, SP, NSP-P4 and SP-P4. (FIG. 5B) The combination of ligands 1 and 2 leads to increased affinity due to the chelate-based avidity effect. Histogram bins (in order left to right): 2:1 P100-2:P11-1 with NSP, NSP-P4, SP-P4. The Y-axis for FIG. 5B is as disclosed for FIG. 5A.

FIG. 6. Bar graph depicting that phage-based ELISA demonstrates the effect of smaller PEG polymers applied as spacers to optimize the geometry of the PEGylated dual ligand combination of P100_{SP}-P4-2+P100_{SP}-P4-1. The dual ligand combination on phage was assayed with and without PEG spacers. Histogram bins (in order left to right): No cells, PEG45, 2:1 P100_{SP}-P4-2:P100_{SP}-P4-1 with No spacer, K14-alkyne (SEQ ID NO:1), PEG7, PEG22, and PEG45.

FIG. 7. A dose response curve demonstrates the specificity of PSMA detection on two types of LNCaP cells relative to the PSMA-negative PC3 cells, as shown by cell-based ELISA. The dual ligand combination of P100_{SP}-P4-2+P100_{SP}-P4-1 and the PEG7 spacer on phage was used for specific detection of PSMA on the cell surface.

FIG. 8. Bar graph depicting a sandwich ELISA demonstrating capture of PSMA positive cells by the dual ligand combination of P100_{SP}-P4-2+P100_{SP}-P4-1 and the PEG7 spacer on phage, which are immobilized on the microtiter plate. Controls are shown in gold color. ‘Media’ indicates fresh culture media, whereas ‘sup’ indicates cell culture supernatant.

FIG. 9. Phage-based ELISA demonstrating unacceptably high non-specific adhesion of phage to the surface of LNCaP cells. Phage-2 display the PSMA ligand 2. Control phage provides a negative control with no ligand displayed on the phage. Throughout this report, error bars for ELISA data represent standard error (n=3). All experimental data points include such error bars, though often these are quite small.

FIG. 10. Cu^{I}-catalyzed azide-alkyne cycloaddition reaction for the generation of oligolysine-PEG wrappers.

FIG. 11. MALDI-TOF characterization of K₁₄-alkyne fused to azide-functionalized PEG22. The data obtained for PEGylated oligolysine showed a characteristic shift in the polydispersed PEG spectra by the expected mass of K₁₄-alkyne (SEQ ID NO:1) (1891.57).

FIG. 12. MALDI-TOF characterization of K₁₄-alkyne (SEQ ID NO:1) fused to azide-functionalized PEG22. The data obtained for PEGylated oligolysine showed a characteristic shift in the polydispersed PEG spectra by the expected mass of K₁₄-alkyne (SEQ ID NO:1) (1891.57).

FIG. 13. Phage-based ELISA demonstrating the ineffectiveness of wrapping phage with PEG polymers due to the encapsulation of oligolysine side chains. In this experiment, phage without wrapper and phage wrapped with PEG7, 22 or 45 at 5 μM concentrations were compared.

FIG. 14. Bar graph depicting that phage-based ELISA illustrates a modest increase in binding affinity using PEGylated ligands on phage targeting LNCaP cells. PEGylated ligands on phage were further engineered for higher affinity recognition.

FIG. 15. Synthesis scheme for the generation of PEGylated ligands on phage through the specific attachment mode. Sequence legend: C-K₁₄ (SEQ ID NO:3).

FIG. 16. Phage-based ELISA demonstrating the significance of the free N-terminus of peptide-2 for PSMA binding as shown by the higher affinity of phage-displayed peptide-2. Synthesis and wrapping of oligolysine-peptide-2 leads to the inversion of geometry providing a free C-terminus.

FIG. 17. Schematic representation of the two constitutional isomers of P100-P4-X. The differences between the products obtained through the specific attachment mode (left) and the non-specific attachment mode (right) are illustrated. The non-specific attachment mode leads to partially modified Glu sidechains, along with a flexible PEG4 linker. In contrast, the specific attachment mode provides the free Glu side chain with a sandwiched PEG4 linker. The abbreviation ‘Alk’ in this schematic represents the alkyne group.

FIG. 18. Bar graph depicting phage-based ELISA demonstrating immobilization of phage in the wells of a microtiter plate, as used in the sandwich ELISA assay (FIG. 8). The wells were incubated with 100 μL/well of 10 nM phage, and then a BSA blocking solution was used. Levels of bound phage were quantified using a horseradish peroxidase-conjugated anti-M13 antibody. The negative control has identical conditions without the addition of phage.

FIG. 19. Representative reverse-phase HPLC for purification of the concentrated reaction mixture to form P100_{SP}-P4-1. To obtain pure P100_{SP}-P4-1, the peak designated in the trace was collected and accurate mass determination was performed using gel permeation chromatography. As shown here, the use of 3K and 5K MW cut off concentrators and extensive washing removed a majority of the unreacted starting materials from the reaction mixture.

FIG. 20. Representative reverse-phase HPLC for purification of the concentrated reaction mixture for P100_{SP}-P4-2. As described above, gel permeation chromatography characterized the mass of this PEGylated ligand. As shown here, the use of 3K and 5K MW cut off concentrators and extensive washing removed a majority of the unreacted starting materials from the reaction mixture.

FIG. 21. Representative reverse-phase HPLC analysis of the purified K₁₄-alkyne. Accurate mass determination was performed using MALDI-TOF mass spectrometry. The features in the HPLC chromatogram before the two minute mark are also observed when only water is injected onto the HPLC column; thus, these features do not reflect the purity of the peptide.

### DETAILED DESCRIPTION

### Definitions

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched non-cyclic carbon chain (or carbon), or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched non-cyclic chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P).

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, non-aromatic cyclic versions of “alkyl” and “heteroalkyl,” respectively, wherein the carbons making up the ring or rings do not necessarily need to be bonded to a hydrogen due to all carbon valencies participating in bonds with non-hydrogen atoms. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, 3-hydroxy-cyclobut-3-enyl-1,2, dione, 1H-1,2,4-triazolyl-5(4H)-one, 4H-1,2,4-triazolyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. A heterocycloalkyl moiety may include one ring heteroatom (e.g., O, N, S, Si, or P). A heterocycloalkyl moiety may include two optionally different ring heteroatoms (e.g., O, N, S, Si, or P). A heterocycloalkyl moiety may include three optionally different ring heteroatoms (e.g., O, N, S, Si, or P). A heterocycloalkyl moiety may include four optionally different ring heteroatoms (e.g., O, N, S, Si, or P). A heterocycloalkyl moiety may include five optionally different ring heteroatoms (e.g., O, N, S, Si, or P). A heterocycloalkyl moiety may include up to 8 optionally different ring heteroatoms (e.g., O, N, S, Si, or P).

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. Non-limiting examples of aryl and heteroaryl groups include pyridinyl, pyrimidinyl, thiophenyl, thienyl, furanyl, indolyl, benzoxadiazolyl, benzodioxolyl, benzodioxanyl, thianaphthanyl, pyrrolopyridinyl, indazolyl, quinolinyl, quinoxalinyl, pyridopyrazinyl, quinazolinonyl, benzoisoxazolyl, imidazopyridinyl, benzofuranyl, benzothienyl, benzothiophenyl, phenyl, naphthyl, biphenyl, pyrrolyl, pyrazolyl, imidazolyl, pyrazinyl, oxazolyl, isoxazolyl, thiazolyl, furylthienyl, pyridyl, pyrimidyl, benzothiazolyl, purinyl, benzimidazolyl, isoquinolyl, thiadiazolyl, oxadiazolyl, pyrrolyl, diazolyl, triazolyl, tetrazolyl, benzothiadiazolyl, isothiazolyl, pyrazolopyrimidinyl, pyrrolopyrimidinyl, benzotriazolyl, benzoxazolyl, or quinolyl. The examples above may be substituted or unsubstituted and divalent radicals of each heteroaryl example above are non-limiting examples of heteroarylene. A heteroaryl moiety may include one ring heteroatom (e.g., O, N, or S). A heteroaryl moiety may include two optionally different ring heteroatoms (e.g., O, N, or S). A heteroaryl moiety may include three optionally different ring heteroatoms (e.g., O, N, or S). A heteroaryl moiety may include four optionally different ring heteroatoms (e.g., O, N, or S). A heteroaryl moiety may include five optionally different ring heteroatoms (e.g., O, N, or S). An aryl moiety may have a single ring. An aryl moiety may have two optionally different rings. An aryl moiety may have three optionally different rings. An aryl moiety may have four optionally different rings. A heteroaryl moiety may have one ring. A heteroaryl moiety may have two optionally different rings. A heteroaryl moiety may have three optionally different rings. A heteroaryl moiety may have four optionally different rings. A heteroaryl moiety may have five optionally different rings.

A fused ring heterocycloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

Each of the above terms (e.g., “alkyl,” “heteroalkyl,”, “cycloalkyl”, “heterocycloalkyl”, “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″, —NR—C(NR′R″)═NR′″, —S(O)R′, —S(O)₂R′, —S(O)₂NR′R″, —NRSO₂R′, —NR′NR″R′″, —ONR′R″, —NR′C═(O)NR″NR′″R″″, —CN, —NO₂, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O) NR′R″, —NR″C(O)R′, —NR′—C(O)NR″R′″, —NR″C(O)₂R′, —NR—C(NR′R″R′″)═NR″″, —NR—C(NR′R″)═N R′″, —S(O)R′, —S(O)₂R′, —S(O)₂NR′R″, —NRSO₂R′, —NR′NR″R′″, —ONR′R″, —NR′C═(O)NR″NR′″R″″, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), and silicon (Si).

A “substituent group,” as used herein, means a group selected from the following moieties:
(A) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, heteroaryl, substituted with at least one substituent selected from:
   (i) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, heteroaryl, substituted with at least one substituent selected from:
      (a) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, heteroaryl, substituted with at least one substituent selected from: oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope disclosed herein.

The symbol “ [symbol] ” denotes the point of attachment of a chemical moiety to the remainder of a molecule or chemical formula.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different.

Descriptions of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

“Nucleic acid” refers to deoxyribonucleotides or ribonucleotides and polymers thereof in either single-, double- or multiple-stranded form, or complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including nucleic acids with a phosphothioate backbone can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism. An “inhibitory nucleic acid” is a nucleic acid (e.g. DNA, RNA, polymer of nucleotide analogs) that is capable of binding to a target nucleic acid (e.g. an mRNA translatable into a protein) and reducing transcription of the target nucleic acid (e.g. mRNA from DNA) or reducing the translation of the target nucleic acid (e.g. mRNA) or altering transcript splicing (e.g. single stranded morpholino oligo).

A “labeled nucleic acid or oligonucleotide” is one that is bound, either covalently, through a linker or a chemical bond, or noncovalently, through ionic, van der Waals, electrostatic, or hydrogen bonds to a label such that the presence of the nucleic acid may be detected by detecting the presence of the detectable label bound to the nucleic acid. Alternatively, a method using high affinity interactions may achieve the same results where one of a pair of binding partners binds to the other, e.g., biotin, streptavidin. In embodiments, the phosphorothioate nucleic acid or phosphorothioate polymer backbone includes a detectable label, as disclosed herein and generally known in the art.

The term “probe” or “primer”, as used herein, is defined to be one or more nucleic acid fragments whose specific hybridization to a sample can be detected. A probe or primer can be of any length depending on the particular technique it will be used for. For example, PCR primers are generally between 10 and 40 nucleotides in length, while nucleic acid probes for, e.g., a Southern blot, can be more than a hundred nucleotides in length. The probe may be unlabeled or labeled as described below so that its binding to the target or sample can be detected. The probe can be produced from a source of nucleic acids from one or more particular (preselected) portions of a chromosome, e.g., one or more clones, an isolated whole chromosome or chromosome fragment, or a collection of polymerase chain reaction (PCR) amplification products. The length and complexity of the nucleic acid fixed onto the target element is not critical to the invention. One of skill can adjust these factors to provide optimum hybridization and signal production for a given hybridization procedure, and to provide the required resolution among different genes or genomic locations.

The probe may also be isolated nucleic acids immobilized on a solid surface (e.g., nitrocellulose, glass, quartz, fused silica slides), as in an array. In some embodiments, the probe may be a member of an array of nucleic acids as described, for instance, in WO 96/17958. Techniques capable of producing high density arrays can also be used for this purpose (see, e.g., Fodor (1991) Science 767-773; Johnston (1998) Curr. Biol. 8: R171-R174; Schummer (1997) Biotechniques 23: 1087-1092; Kern (1997) Biotechniques 23: 120-124; U.S. Pat. No. 5,143,854).

The words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “purified” denotes that a nucleic acid or protein gives rise to essentially one band in an electrophoretic gel. In some embodiments, the nucleic acid or protein is at least 50% pure, optionally at least 65% pure, optionally at least 75% pure, optionally at least 85% pure, optionally at least 95% pure, and optionally at least 99% pure.

The term “isolated” may also refer to a cell or sample cells. An isolated cell or sample cells are a single cell type that is substantially free of many of the components which normally accompany the cells when they are in their native state or when they are initially removed from their native state. In certain embodiments, an isolated cell sample retains those components from its natural state that are required to maintain the cell in a desired state. In some embodiments, an isolated (e.g. purified, separated) cell or isolated cells, are cells that are substantially the only cell type in a sample. A purified cell sample may contain at least 60%, 70%, 75%, 80%, 85%, 90%, 95%, 96%, 97%, 98%, 99%, or 100% of one type of cell. An isolated cell sample may be obtained through the use of a cell marker or a combination of cell markers, either of which is unique to one cell type in an unpurified cell sample. In some embodiments, the cells are isolated through the use of a cell sorter. In some embodiments, antibodies against cell proteins are used to isolate cells.

As used herein, the term “conjugate” refers to the association between atoms or molecules. The association can be direct or indirect. For example, a conjugate between a polymer and a ligand or recognition moiety provided herein can be direct, e.g., by covalent bond, or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, conjugates are formed using conjugate chemistry including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the polymer is non-covalently attached to the ligand through a non-covalent chemical reaction between a component of the polymer and a component of the ligand. In other embodiments, the polymer is covalently bound to the ligand or recognition moiety using a covalent linker, wherein the covalent linker is attached to the polymer at one end and to the ligand or recognition moiety at the other end. The linker attachment to the polymer or to the ligand or recognition moiety may be accomplished using one or more reactive moieties, e.g., bioconjugate techniques, a covalent reactive moiety, as described herein (e.g., alkyne, azide, maleimide or thiol reactive moiety).

Useful reactive moieties or functional groups (chemical reactive functional groups) used for conjugate chemistries (click chemistries) herein include, for example:

(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;

(b) hydroxyl groups which can be converted to esters, ethers, aldehydes, etc.

(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;

(d) dienophile groups which are capable of participating in Diels-Alder reactions such as, for example, maleimido groups;

(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;

(f) sulfonyl halide groups for subsequent reaction with amines, for example, to form sulfonamides;

(g) thiol groups, which can be converted to disulfides, reacted with acyl halides, or bonded to metals such as gold;

(h) amine or sulfhydryl groups, which can be, for example, acylated, alkylated or oxidized;

(i) alkenes, which can undergo, for example, cycloadditions, acylation, Michael addition, and the like;

(j) epoxides, which can react with, for example, amines and hydroxyl compounds;

(k) phosphoramidites and other standard functional groups useful in nucleic acid synthesis;

(l) metal silicon oxide bonding;

(m) metal bonding to reactive phosphorus groups (e.g. phosphines) to form, for example, phosphate diester bonds; and

(n) sulfones, for example, vinyl sulfone.

Chemical synthesis of compositions by joining modular units using conjugate (click) chemistry may also be sued to attach the covalent linker to the polymer and/or to the ligand or recognition moiety, which is well known in the art and described, for example, in H. C. Kolb, M. G. Finn and K. B. Sharpless ((2001). “Click Chemistry: Diverse Chemical Function from a Few Good Reactions”. Angewandte Chemie International Edition 40 (11): 2004-2021); R. A. Evans ((2007). “The Rise of Azide-Alkyne 1,3-Dipolar ‘Click’ Cycloaddition and its Application to Polymer Science and Surface Modification”. Australian Journal of Chemistry 60 (6): 384-395; W. C. Guida et al. Med. Res. Rev. p 3 1996; Spiteri, Christian and Moses, John E. ((2010). “Copper-Catalyzed Azide-Alkyne Cycloaddition: Regioselective Synthesis of 1,4,5-Trisubstituted 1,2,3-Triazoles”. Angewandte Chemie International Edition 49 (1): 31-33); Hoyle, Charles E. and Bowman, Christopher N. ((2010). “Thiol-Ene Click Chemistry”. Angewandte Chemie International Edition 49 (9): 1540-1573); Blackman, Melissa L. and Royzen, Maksim and Fox, Joseph M. ((2008). “Tetrazine Ligation: Fast Bioconjugation Based on Inverse-Electron-Demand Diels-Alder Reactivity”. Journal of the American Chemical Society 130 (41): 13518-13519); Devaraj, Neal K. and Weissleder, Ralph and Hilderbrand, Scott A. ((2008). “Tetrazine Based Cycloadditions: Application to Pretargeted Live Cell Labeling”. Bioconjugate Chemistry 19 (12): 2297-2299); Stöckmann, Henning; Neves, Andre; Stairs, Shaun; Brindle, Kevin; Leeper, Finian ((2011). “Exploring isonitrile-based click chemistry for ligation with biomolecules”. Organic & Biomolecular Chemistry), all of which are hereby incorporated by reference in their entirety and for all purposes.

The reactive functional groups can be chosen such that they do not participate in, or interfere with, the chemical stability of the proteins described herein. By way of example, the polymer or ligand/recognition moiety can include a vinyl sulfone or other reactive moiety (e.g., maleimide). Optionally, the polymer or ligand can include a reactive moiety having the formula S—S—R. R can be, for example, a protecting group. Optionally, R is hexanol. As used herein, the term hexanol includes compounds with the formula C₆H₁₃OH and includes, 1-hexanol, 2-hexanol, 3-hexanol, 2-methyl-1-pentanol, 3-methyl-1-pentanol, 4-methyl-1-pentanol, 2-methyl-2-pentanol, 3-methyl-2-pentanol, 4-methyl-2-pentanol, 2-methyl-3-pentanol, 3-methyl-3-pentanol, 2,2-dimethyl-1-butanol, 2,3-dimethyl-1-butanol, 3,3-dimethyl-1-butanol, 2,3-dimethyl-2-butanol, 3,3-dimethyl-2-butanol, and 2-ethyl-1-butanol. Optionally, R is 1-hexanol.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. The terms apply to macrocyclic peptides, peptides that have been modified with non-peptide functionality, peptidomimetics, polyamides, and macrolactams. A “fusion protein” refers to a chimeric protein encoding two or more separate protein sequences that are recombinantly expressed as a single moiety.

The term “peptidyl” and “peptidyl moiety” means a monovalent peptide.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross-reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

A “labeled protein or polypeptide” is one that is bound, either covalently, through a linker or a chemical bond, or noncovalently, through ionic, van der Waals, electrostatic, or hydrogen bonds to a label such that the presence of the labeled protein or polypeptide may be detected by detecting the presence of the label bound to the labeled protein or polypeptide. Alternatively, methods using high affinity interactions may achieve the same results where one of a pair of binding partners binds to the other, e.g., biotin, streptavidin.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells.

The term “cell surface marker” as used herein, refers to a protein or a group of proteins expressed on the surface of cells that serve as markers of specific cell types.

The term “polymer” or “polymers” as provided herein refers to synthetic or natural molecules, or macromolecules, composed of multiple repeated subunits (monomers). Synthetic polymers (e.g., synthetic plastics such as polystyrene) and natural biopolymers (e.g., DNA, proteins) may be distinguished. Polymers, both natural and synthetic, are created via polymerization of many small molecules, known as monomers. In embodiments, polymers have a large molecular mass relative to small molecule compounds and, therefore, produce unique physical properties (e.g., toughness, viscoelasticity, tendency to form glasses and semicrystalline structures). In embodiments, the polymers are charged (charged polymers). The charged polymers provided herein may include a positive charge or a negative charge. Thus, in embodiments, the charged polymer is an anionic polymer. In embodiments, the charged polymer is a cationic polymer. Non-limiting examples of polymers useful for the compositions and methods provided herein include gum arabic, gum acacia, gum tragacanth, locust bean gum, guar gum, hydroxypropyl guar, xanthan gum, talc, cellulose gum, sclerotium gum, carageenan gum, karaya gum, cellulose gum, rosin, methylcellulose, hydroxyethylcellulose, hydroxypropylcellulose, hydroxymethylcellulose, hydroxypropylmethylcellulose, methylhydroxyethylcellulose, cetyl hydroxyethylcellulose, carboxymethylcellulose, corn starch, hydroxypropyl starch phosphate, distarch phosphate, distarch dimethylene urea, aluminum starch octenyl succinate, maltodextrin, dextran, poly(acrylamide), PEG-150 distearate, PEG-150/decyl alcohol/SMDI copolymer, PEG-150/stearyl alcohol/SMDI copolymer, PEG-180/Laureth-50/TMMG copolymer, Polyether 1, acrylic acid/acrylamidomethyl propane sulfonic acid copolymer, acrylate/C10-30 alkyl acrylate cross polymer, acrylate/beheneth-25 methacrylate copolymer, acrylate/steareth-20 methacrylate copolymer, acrylate/steareth-20 copolymer, acrylate/VA cross polymer, acrylic acid/acrylonitrogen copolymer, ammonium acryloyldimethyltaurate/beheneth-25 methacrylate copolymer, ammonium acryloyldimethyltaurate/VP copolymer, sodium acrylate copolymer, PVM/MA decadiene cross polymer, alginic acid, propylene glycol alginate, dimethicone, silica dimethyl silylate, a dimethylacrylamide/acrylic acid/polystyrene ethyl methacrylate copolymer, PLGA polymer, polylactide, polyethylene glycol, carbomer, trolamine, derivatives thereof, and mixtures thereof. In embodiments, the polyethylene glycol is PEG3380. PEG3380 refers, in the customary sense, to CAS Registry No. 71767-64-1. In embodiments, the carbomer is CARBOPOL® 980. The term “carbomer” refers to cross linked polyacrylate polymers as known in the art and, for example, to CARBOPOL® 980 or CARBOPOL® 980 polymer, which are defined by CAS Registry Nos. 9063-87-0, 9003-01-4, or 600-07-7, respectively. The polyacrylate polymer may be, but is not limited to, poly-2-methylbutanoic acid, poly-prop-2-enoic acid, polyacrylic acid.

In embodiments, the polymer is a block polymer. In embodiments, the block polymer is a Lysine₁₄ block polymer. A Lysine₁₄ block polymer (“K₁₄”) as provided herein refers to a polymer derived from lysine homopolymer subunits (monomers), which are linked by covalent bonds.

A “solid support” as provided herein refers to any appropriate material that can be modified to contain discrete individual sites for the attachment or association of an electronically conductive polymer as provided herein including embodiments thereof and is amenable to the methods provided herein including embodiments thereof. Examples of solid supports include without limitation, glass and modified or functionalized glass (e.g., carboxymethyldextran functionalized glass), plastics (including acrylics, polystyrene and copolymers of styrene and other materials, polypropylene, polyethylene, polybutylene, polyurethanes, Teflon™, etc.), polysaccharides, nylon or nitrocellulose, composite materials, ceramics, and plastic resins, silica or silica-based materials including silicon and modified silicon (e.g., patterned silicon), carbon, metals, quartz (e.g., patterned quartz), inorganic glasses, plastics, optical fiber bundles, and a variety of other polymers (e.g., electronically conductive polymers such as poly-3,4-ethylenedioxythiophene, PEDOT). In general, the solid support allows optical detection and do not appreciably fluoresce. The solid support may be planar (e.g., flat planar substrates such as glass, polystyrene and other plastics and acrylics). Although it will be appreciated by a person of ordinary skill in the art that other configurations of solid supports may be used as well; for example, three dimensional configurations can be used. The solid support may be modified to contain discrete, individual sites (also referred to herein as “wells”) for polymer binding. These sites generally include physically altered sites, i.e. physical configurations such as wells or small depressions in the substrate that can retain the polymers. The wells may be formed using a variety of techniques well known in the art, including, but not limited to, photolithography, stamping techniques, molding techniques and microetching techniques. It will be appreciated by a person of ordinary skill in the art that the technique used will depend on the composition and shape of the solid support. In embodiments, physical alterations are made in a surface of the solid support to produce wells. In embodiments, the solid support is a microtiter plate.

The term “recognition moiety” or “ligand” (also referred to herein as a ligand domain) refers to a composition (e.g., atom, molecule, ion, molecular ion, compound, particle, protein, peptide, nucleic acid, oligosaccharide, polysaccharide, or small molecule) capable of binding (e.g. specifically binding) to a second complementary ligand-binding composition (e.g., analyte, polymer, protein, marker, small molecule, ligand, polysaccharide, aptamer, or other binder) to form a complex. A recognition moiety as provided herein may without limitation bind to biomolecules (e.g., hormones, cytokines, proteins, nucleic acids, lipids, carbohydrates, cellular membrane antigens and receptors (neural, hormonal, nutrient, and cell surface receptors or their ligands)); whole cells or lysates thereof (e.g., prokaryotic (e.g., pathogenic bacteria), eukaryotic cells (e.g., mammalian tumor cells); viruses (e.g., retroviruses, herpesviruses, adenoviruses, lentiviruses and spores); chemicals (e.g., solvents, polymers, organic materials, small molecules); therapeutic molecules (e.g., therapeutic drugs, abused drugs, antibiotics); environmental pollutants (e.g., pesticides, insecticides, toxins). In embodiments, the recognition moiety is a cell surface marker binding moiety (i.e., a composition that recognizes and binds to a cell surface marker). In embodiments, the recognition moiety is a polypeptide. In embodiments, the recognition moiety is an antibody or a fragment thereof.

As used herein, the terms “specific binding” or “specifically binds” refer to two molecules forming a complex that is relatively stable under physiologic conditions.

Methods for determining whether a ligand binds to a protein and/or the affinity for a ligand to a protein are known in the art. For example, the binding of a ligand to a protein can be detected and/or quantified using a variety of techniques such as, but not limited to, Western blot, dot blot, surface plasmon resonance method (e.g., BIAcore system; Pharmacia Biosensor AB, Uppsala, Sweden and Piscataway, N.J.), isothermal titration calorimetry (ITC), or enzyme-linked immunosorbent assays (ELISA).

Immunoassays which can be used to analyze immunospecific binding and cross-reactivity of the ligand include, but are not limited to, competitive and non-competitive assay systems using techniques such as Western blots, RIA, ELISA (enzyme linked immunosorbent assay), “sandwich” immunoassays, immunoprecipitation assays, immunodiffusion assays, agglutination assays, complement-fixation assays, immunoradiometric assays, and fluorescent immunoassays. Such assays are routine and well known in the art.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable heavy chain,” “V_{H},” or “VH” refer to the variable region of an immunoglobulin heavy chain, including an Fv, scFv, dsFv or Fab; while the terms “variable light chain,” “V_{L}” or “VL” refer to the variable region of an immunoglobulin light chain, including of an Fv, scFv, dsFv or Fab.

Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (see, e.g., FUNDAMENTAL IMMUNOLOGY (Paul ed., 4th ed. 2001). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al. (1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301.

The term “diagnosis” refers to a relative probability that a disease (e.g. cancer, urinary tract infection, infection, or other disease) is present in the subject. Similarly, the term “prognosis” refers to a relative probability that a certain future outcome may occur in the subject with respect to a disease state. For example, in the context of the present invention, prognosis can refer to the likelihood that an individual will develop a disease (e.g. cancer, urinary tract infection, infection, or other disease), or the likely severity of the disease (e.g., duration of disease). The terms are not intended to be absolute, as will be appreciated by any one of skill in the field of medical diagnostics.

As used herein, a “diagnostically effective amount” of a composition described herein is an amount sufficient to produce a clinically useful characterization or measurement of a disease state, such as an infection or cancer, (e.g. in an individual, patient, human, mammal, clinical sample, tissue, biopsy). A clinically useful characterization or measurement of a disease state, such as an infection or cancer, (e.g. in an individual, patient, human, mammal, clinical sample, tissue, biopsy) is one containing sufficient detail to enable an experienced clinician to assess the degree and/or extent of disease for purposes of diagnosis, monitoring the efficacy of a therapeutic intervention, and the like.

The terms “disease” or “condition” refer to a state of being or health status of a patient or subject capable of being treated with a compound, pharmaceutical composition, or method provided herein. In embodiments, the disease is cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma).

The compounds disclosed herein may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds disclosed herein, whether radioactive or not, are encompassed within the scope disclosed herein.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating,” and conjugations thereof, include prevention of an injury, pathology, condition, or disease.

An “effective amount” is an amount sufficient to accomplish a stated purpose (e.g., achieve the effect for which it is administered, treat a disease, reduce tumor size, and the like). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Subject,” “patient,” “subject in need thereof” and the like refer to a living organism suffering from or prone to a disease or condition that can be treated by administration of a compound or pharmaceutical composition, as provided herein. Non-limiting examples include humans, other mammals, bovines, rats, mice, dogs, monkeys, goat, sheep, cows, deer, and other non-mammalian animals. In embodiments, a subject is human.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Unless indicated to the contrary, the terms “active agent,” “active ingredient,” “therapeutically active agent,” “therapeutic agent” and like are used synonymously. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, polyethylene glycol, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

As used herein, the term “administering” means oral administration, administration as an inhaled aerosol or as an inhaled dry powder, suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example corticosteroids, antibiotics, cancer therapies such as chemotherapy, hormonal therapy, radiotherapy, or immunotherapy. The compound of the invention can be administered alone or can be coadministered to the patient. Co-administration is meant to include simultaneous or sequential administration of the compound individually or in combination (more than one compound or agent). Thus, the preparations can also be combined, when desired, with other active substances (e.g., to reduce metabolic degradation, to promote the penetration of tissues, or the like). The compositions of the present invention can be delivered transdermally, by a topical route, formulated as applicator sticks, solutions, suspensions, emulsions, gels, creams, ointments, nanoparticles, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

Pharmaceutical compositions provided by the present invention include compositions wherein the active ingredient (e.g., conjugated wrapping phages described herein, including embodiments or examples) is contained in a therapeutically effective amount, i.e., in an amount effective to achieve its intended purpose. The actual amount effective for a particular application will depend, inter alia, on the condition being treated. Determination of a therapeutically effective amount of a compound of the invention is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art.

As used herein, the term “cancer” refers to all types of cancer, neoplasm or malignant tumors found in mammals, including glioblastomas, leukemias, lymphomas, melanomas, neuroendocrine tumors, carcinomas and sarcomas. Exemplary cancers that may be treated with a compound, pharmaceutical composition, or method provided herein include glioblastoma, lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound, pharmaceutical composition, or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound, pharmaceutical composition, or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound, pharmaceutical composition, or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound, pharmaceutical composition, or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, ductal carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lobular carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tubular carcinoma, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

“Anti-cancer agent” is used in accordance with its plain ordinary meaning and refers to a composition (e.g. compound, drug, antagonist, inhibitor, modulator) having antineoplastic properties or the ability to inhibit the growth or proliferation of cells.

The term “associated” or “associated with” in the context of a substance or substance activity or function associated with a disease (e.g., diabetes, cancer (e.g. prostate cancer, renal cancer, metastatic cancer, melanoma, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma)) means that the disease (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma) is caused by (in whole or in part), or a symptom of the disease is caused by (in whole or in part) the substance or substance activity or function.

Overview

The migration and dissemination of tumor cells, termed metastasis, causes about 90% of cancer deaths. Metastasis requires loss of apoptotic regulation, and such cells respond poorly to conventional anti-cancer treatments. Specific detection of circulating tumor cells and characterization of their aggressiveness could improve cancer diagnostics and treatment. Chemically modified viruses (such as phage) could provide an inexpensive and efficient approach to detect tumor cells and quantitate their cell surface biomarkers.

The M13 virus consists of a circular, single-stranded DNA genome surrounded by a protein coat composed of approximately 2700 copies of the major coat protein, P8, an α-helical protein of 50 amino acid residues with an unstructured N-terminus. One Glu and two Asp residues near the N-terminus of P8 impart a high negative charge to the outer surface of the virus at physiological pH. Selections with phage-displayed libraries of peptides and proteins can target tissue-cultured cells and even organs in living organisms. Phage has also been incorporated into nanomedicine platforms for targeted drug delivery and imaging. Such applications require low background binding by phage to cell surfaces.

Phage typically adheres to cell surfaces with high affinity. Unfortunately, such non-specific adhesion complicates the design of phage-based sensors for the detection of tumor cells: the non-specific background can reduce the signal to noise ratios and the ability to distinguish tumor from non-tumor cells.

Provided herein are compositions and methods that solve this and other problems. In embodiments, a viral composition (e.g., a conjugated wrapped phage) is provided having a surface (e.g., an engineered surface) wrapped with a polymer. The polymer may be a variety of different covalent linker architectures (e.g., oligolysine, linkers, spacers, and recognition moieties). As demonstrated herein, the viral composition described herein may be used to specifically detect cancer cells expressing cancer biomarkers. In embodiments, this approach also allows quantification of biomarker levels on the cell surface, and can distinguish more aggressive forms of the disease. In embodiments, the viral composition described herein results in more than 75% reduction of the non-specific adhesion of virus to cell surfaces.

Compositions

In one aspect, provided herein is a viral composition. The viral composition includes (a) a whole viral particle comprising a charged protein coat that has a charged coat protein; (b) a first polymer electrostatically bound to the charged coat protein; and (c) a covalent linker linking the first polymer to a recognition moiety. In embodiments, the viral composition is a conjugated wrapped phage.

The term “conjugated wrapped phage” and the like as used herein means a bacteriophage (also referred to herein as a phage) which is in contact with a charged polymer which at least partially encircles or enfolds the phage charged protein coat as disclosed herein. In embodiments, the polymer in this context binds the phage non-covalently via e.g., electrostatic attraction between the charged protein coat of the phage and charges on the polymer. The charged protein coat includes a plurality of charged coat proteins in contact with the charged polymer. The plurality of charged coat proteins has the opposite charge of the charged polymer. In embodiments, a phage described herein is in contact with a charged polymer (e.g., a first polymer) that fully encircles or enfolds the phage charged protein coat as disclosed herein. In embodiments, the charged polymer (e.g., a first polymer) encircles 2, 3, 4, 5, 6, 7, 8, 9, 10, 11, 12, 13, 14, 15, 16, 17, 18, 19, 20 or more times the charged protein coat.

The term “whole viral particle” as used herein, refers to a complete viral particle that includes the genetic material made from either DNA or RNA and a protein coat, also called the capsid, which surrounds and protects the genetic material. In embodiments, where appropriate, the whole viral particle includes; an envelope of lipids that surrounds the protein coat (e.g. when the viral particle is outside a cell). A protein coat or a capsid is the protein shell of a virus. A charged protein coat, as used herein, refers to a protein coat having either a net positive or a net negative electric charge. In embodiments, a charged protein coat has a net negative electric charge.

In embodiments, the whole viral particle is a whole bacteriophage (or phage) that includes the genetic material made from either DNA or RNA and a protein coat, also called the capsid, which surrounds and protects the genetic material. A protein coat or a capsid is the protein shell of the phage. A charged protein coat, as used herein, refers to a protein coat having either a net positive or a net negative electric charge. In embodiments, a charged protein coat has a net negative electric charge.

A coat protein, as used herein, refers to a protein within the capsid (or protein coat). A charged coat protein refers to a coat protein having either a net positive or a net negative electric charge. In embodiments, a charged coat protein has a net negative electric charge.

The term “covalent linker,” “linker,” “spacer” are used herein interchangeably and refer to a divalent chemical moiety attached at each end to the remainder of the compound. In embodiments, the covalent linker is -L¹-L²-L³-L⁴-L⁵-L⁶-.

In embodiments, L¹ is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl or -L^{1A}-L^{1B}-L^{1C}-L^{1D}-L^{1E}-L^{1F}-L^{1G}-L^{1H}-L^{1I}-L^{1J}-.

In embodiments, L^{1A} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{1B} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{1C} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{1d} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{1E} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{1F} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{1G} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{1H} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{1I} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{1J} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, at least one (e.g. 1, 2, 3 or 4) of L^{1A}, L^{1B}, L^{1C}, L^{1D}, L^{1E}, L^{1F}, L^{1G}, L^{1H}, L^{1I}, and L^{1J} is not a bond

In embodiments, at least one (e.g. 1, 2, 3 or 4) of L^{1A}, L^{1B}, L^{1C}, L^{1D}, L^{1E}, L^{1F}, L^{1G}, L^{1H}, L^{1I}, and L^{1J} is a cleavable linker.

In embodiments, L² is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl or -L^{2A}-L^{2B}-L^{2C}-L^{2D}-L^{2E}-L^{2F}-L^{2G}-L^{2H}-L^{2I}-L^{2J}-.

In embodiments, L^{2A} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{2B} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{2C} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{2D} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{2E} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{2F} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{2G} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{2H} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{2I} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{2J} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, at least one (e.g. 1, 2, 3 or 4) of L^{2A}, L^{2B}, L^{2C}, L^{2D}, L^{2E}, L^{2F}, L^{2G}, L^{2H}, L^{2I}, and L^{2J} is not a bond.

In embodiments, at least one (e.g. 1, 2, 3 or 4) of L^{2A}, L^{2B}, L^{2C}, L^{2D}, L^{2E}, L^{2F}, L^{2G}, L^{2H}, L^{2I}, and L^{2J} is a cleavable linker.

In embodiments, L³ is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl or -L^{3A}-L^{3B}-L^{3C}-L^{3D}-L^{3E}-L^{3F}-L^{3G}-L^{3H}-L^{3I}-L^{3J}.

In embodiments, L^{3A} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{3B} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{3C} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{3D} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{3E} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{3F} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{3G} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{3H} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{3I} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{3J} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, at least one (e.g. 1, 2, 3 or 4) of L^{3A}, L^{3B}, L^{3C}, L^{3D}, L^{3E}, L^{3F}, L^{3G}, L^{3H}, L^{3I}, and L^{3J} is not a bond.

In embodiments, at least one (e.g. 1, 2, 3 or 4) of L^{3A}, L^{3B}, L^{3C}, L^{3D}, L^{3E}, L^{3F}, L^{3G}, L^{3H}, L^{3I}, and L^{3J} is a cleavable linker.

In embodiments, L⁴ is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl or -L^{4A}-L^{4B}-L^{4C}-L^{4D}-L^{4E}-L^{4F}-L^{4G}-L^{4H}-L^{4I}-L^{4J}-.

In embodiments, L^{4A} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{4B} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{4C} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{4D} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{4E} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{4F} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{4G} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{4H} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{4I} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{4J} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, at least one (e.g. 1, 2, 3 or 4) of L^{4A}, L^{4B}, L^{4C}, L^{4D}, L^{4E}, L^{4F}, L^{4G}, L^{4H}, L^{4I}, and L^{4J} is not a bond.

In embodiments, at least one (e.g. 1, 2, 3 or 4) of L^{4A}, L^{4B}, L^{4C}, L^{4D}, L^{4E}, L^{4F}, L^{4G}, L^{4H}, L^{4I}, and L^{4J} is a cleavable linker.

In embodiments, L⁵ is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl or -L^{5A}-L^{5B}-L^{5C}-L^{5D}-L^{5E}-L^{5F}-L^{5G}-L^{5H}-L^{5I}-L^{5J}-.

In embodiments, L^{5A} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{5B} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{5C} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{5D} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{5E} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{5F} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{5G} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{5H} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{5I} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{5J} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, at least one (e.g. 1, 2, 3 or 4) of L^{5A}, L^{5B}, L^{5C}, L^{5D}, L^{5E}, L^{5F}, L^{5G}, L^{5H}, L^{5I}, and L^{5J} is not a bond.

In embodiments, at least one (e.g. 1, 2, 3 or 4) of L^{5A}, L^{5B}, L^{5C}, L^{5D}, L^{5E}, L^{5F}, L^{5G}, L^{5H}, L^{5I}, and L^{5J} is a cleavable linker.

In embodiments, L⁶ is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl or -L^{6A}-L^{6B}-L^{6C}-L^{6D}-L^{6E}-L^{6F}-L^{6G}-L^{6H}-L^{6I}-L^{6J}-.

In embodiments, L^{6A} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{6B} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{6C} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{6D} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{6E} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{6F} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{6G} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{6H} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{6I} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, L^{6J} is a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

In embodiments, at least one (e.g. 1, 2, 3 or 4) of L^{6A}, L^{6B}, L^{6C}, L^{6D}, L^{6E}, L^{6F}, L^{6G}, L^{6H}, L^{6I}, and L^{6J} is not a bond.

In embodiments, at least one (e.g. 1, 2, 3 or 4) of L^{6A}, L^{6B}, L^{6C}, L^{6D}, L^{6E}, L^{6F}, L^{6G}, L^{6H}, L^{6I}, and L^{6J} is a cleavable linker.

In embodiments, L¹ is substituted or unsubstituted heteroalkyl, L² is substituted or unsubstituted heteroaryl, L³ is substituted or unsubstituted heteroalkyl, L⁴ is substituted or unsubstituted heterocycloalkyl, L⁵ is a substituted or unsubstituted heteroalkyl, and L⁶ is a bond.

In embodiments, L⁴ is

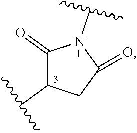 [SMILES: *C1CC(=O)N(*)C1=O]

where the carbon at the 3 position is covalently attached to L⁵.

In embodiments, L⁵ is —S—CH₂—CH(NH₂)—C(O)— or —S—CH₂—CH(C(O)OH)—NH—, wherein the sulfur of L⁵ is attached to L⁴.

In embodiments, L³ comprises a polyethylene glycol linker. In embodiments, polyethylene glycol linker comprises 2 to 150 oxyethylene units (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 oxyethylene units).

In some embodiments, each substituted group described for L¹, L², L³, L⁴, L⁵, L⁶, L^{1A}, L^{1B}, L^{1C}, L^{1D}, L^{1E}, L^{1F}, L^{1G}, L^{1H}, L^{1I}, L^{1J}, L^{2A}, L^{2B}, L^{2C}, L^{2D}, L^{2E}, L^{2F}, L^{2G}, L^{2H}, L^{2I}, L^{2J}, L^{3A}, L^{3B}, L^{3C}, L^{3D}, L^{3E}, L^{3F}, L^{3G}, L^{3H}, L^{3I}, L^{3J}, L^{4A}, L^{4B}, L^{4C}, L^{4D}, L^{4E}, L^{4F}, L^{4G}, L^{4H}, L^{4I}, L^{4J}, L^{5A}, L^{5B}, L^{5C}, L^{5D}, L^{5E}, L^{5F}, L^{5G}, L^{5H}, L^{5I}, L^{5J}, L^{6A}, L^{6B}, L^{6C}, L^{6D}, L^{6E}, L^{6F}, L^{6G}, L^{6H}, L^{6I}, and L^{6J} is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described for L¹, L², L³, L⁴, L⁵, L⁶, L^{1A}, L^{1B}, L^{1C}, L^{1D}, L^{1E}, L^{1F}, L^{1G}, L^{1H}, L^{1I}, L^{1J}, L^{2A}, L^{2B}, L^{2C}, L^{2D}, L^{2E}, L^{2F}, L^{2G}, L^{2H}, L^{2I}, L^{2J}, L^{3A}, L^{3B}, L^{3C}, L^{3D}, L^{3E}, L^{3F}, L^{3G}, L^{3H}, L^{3I}, L^{3J}, L^{4A}, L^{4B}, L^{4C}, L^{4D}, L^{4E}, L^{4F}, L^{4G}, L^{4H}, L^{4I}, L^{4J}, L^{5A}, L^{5B}, L^{5C}, L^{5D}, L^{5E}, L^{5F}, L^{5G}, L^{5H}, L^{5I}, L^{5J}, L^{6A}, L^{6B}, L^{6C}, L^{6D}, L^{6E}, L^{6F}, L^{6G}, L^{6H}, L^{6I}, and L^{6J} is substituted with at least one substituent group.

In some embodiments, each substituted group described for L¹, L², L³, L⁴, L⁵, L⁶, L^{1A}, L^{1B}, L^{1C}, L^{1D}, L^{1E}, L^{1F}, L^{1G}, L^{1H}, L^{1I}, L^{1J}, L^{2A}, L^{2B}, L^{2C}, L^{2D}, L^{2E}, L^{2F}, L^{2G}, L^{2H}, L^{2I}, L^{2J}, L^{3A}, L^{3B}, L^{3C}, L^{3D}, L^{3E}, L^{3F}, L^{3G}, L^{3H}, L^{3I}, L^{3J}, L^{4A}, L^{4B}, L^{4C}, L^{4D}, L^{4E}, L^{4F}, L^{4G}, L^{4H}, L^{4I}, L^{4J}, L^{5A}, L^{5B}, L^{5C}, L^{5D}, L^{5E}, L^{5F}, L^{5G}, L^{5H}, L^{5I}, L^{5J}, L^{6A}, L^{6B}, L^{6C}, L^{6D}, L^{6E}, L^{6F}, L^{6G}, L^{6H}, L^{6I}, and L^{6J} is substituted with at least one lower substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described for L¹, L², L³, L⁴, L⁵, L⁶, L^{1A}, L^{1B}, L^{1C}, L^{1D}, L^{1E}, L^{1F}, L^{1G}, L^{1H}, L^{1I}, L^{1J}, L^{2A}, L^{2B}, L^{2C}, L^{2D}, L^{2E}, L^{2F}, L^{2G}, L^{2H}, L^{2I}, L^{2J}, L^{3A}, L^{3B}, L^{3C}, L^{3D}, L^{3E}, L^{3F}, L^{3G}, L^{3H}, L^{3I}, L^{3J}, L^{4A}, L^{4B}, L^{4C}, L^{4D}, L^{4E}, L^{4F}, L^{4G}, L^{4H}, L^{4I}, L^{4J}, L^{5A}, L^{5B}, L^{5C}, L^{5D}, L^{5E}, L^{5F}, L^{5G}, L^{5H}, L^{5I}, L^{5J}, L^{6A}, L^{6B}, L^{6C}, L^{6D}, L^{6E}, L^{6F}, L^{6G}, L^{6H}, L^{6I}, and L^{6J} is substituted with at least one lower substituent group.

In some embodiments, each substituted group described for L¹, L², L³, L⁴, L⁵, L⁶, L^{1A}, L^{1B}, L^{1C}, L^{1D}, L^{1E}, L^{1F}, L^{1G}, L^{1H}, L^{1I}, L^{1J}, L^{2A}, L^{2B}, L^{2C}, L^{2D}, L^{2E}, L^{2F}, L^{2G}, L^{2H}, L^{2I}, L^{2J}, L^{3A}, L^{3B}, L^{3C}, L^{3D}, L^{3E}, L^{3F}, L^{3G}, L^{3H}, L^{3I}, L^{3J}, L^{4A}, L^{4B}, L^{4C}, L^{4D}, L^{4E}, L^{4F}, L^{4G}, L^{4H}, L^{4I}, L^{4J}, L^{5A}, L^{5B}, L^{5C}, L^{5D}, L^{5E}, L^{5F}, L^{5G}, L^{5H}, L^{5I}, L^{5J}, L^{6A}, L^{6B}, L^{6C}, L^{6D}, L^{6E}, L^{6F}, L^{6G}, L^{6H}, L^{6I}, and L^{6J} is substituted with at least one size-limited substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described for L¹, L², L³, L⁴, L⁵, L⁶, L^{1A}, L^{1B}, L^{1C}, L^{1D}, L^{1E}, L^{1F}, L^{1G}, L^{1H}, L^{1I}, L^{1J}, L^{2A}, L^{2B}, L^{2C}, L^{2D}, L^{2E}, L^{2F}, L^{2G}, L^{2H}, L^{2I}, L^{2J}, L^{3A}, L^{3B}, L^{3C}, L^{3D}, L^{3E}, L^{3F}, L^{3G}, L^{3H}, L^{3I}, L^{3J}, L^{4A}, L^{4B}, L^{4C}, L^{4D}, L^{4E}, L^{4F}, L^{4G}, L^{4H}, L^{4I}, L^{4J}, L^{5A}, L^{5B}, L^{5C}, L^{5D}, L^{5E}, L^{5F}, L^{5G}, L^{5H}, L^{5I}, L^{5J}, L^{6A}, L^{6B}, L^{6C}, L^{6D}, L^{6E}, L^{6F}, L^{6G}, L^{6H}, L^{6I}, and L^{6J} is substituted with at least one size-limited substituent group.

In embodiments, each of the plurality of charged coat proteins is a negatively charged coat protein. In embodiments, each of the plurality of charged coat proteins is a positively charged coat protein. In embodiments, each of the plurality of charged coat proteins includes one or more negatively charged amino acid residues. In embodiments, each of the plurality of charged coat proteins includes one or more Glu or one or more Asp residues. In embodiments, each of the plurality of charged coat proteins includes one or more Glu and one or more Asp residues. In embodiments, the one or more Glu or one or more Asp residues form part of the N-terminus of the charged coat protein. In embodiments, the charged coat protein is P8.

In embodiments, a plurality of charged coat proteins includes at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 or more of charged coat proteins. In embodiments, the charged proteins of the plurality of charged coat proteins are the same. In embodiments, the charged coat proteins plurality of charged coat proteins are not the same.

The term “P8” or “P8 protein” as provided herein includes any of the recombinant or naturally-occurring forms of the viral coat protein P8 or variants or homologs thereof that maintain P8 protein activity (e.g. within at least 50%, 80%, 90%, 95%, 96%, 97%, 98%, 99% or 100% activity compared to P8). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring P8 polypeptide. In embodiments, P8 is the protein as identified by the NCBI sequence reference GI:402239556, homolog or functional fragment thereof.

In embodiments, the whole viral particle is a whole bacteriophage viral particle. In embodiments, the whole viral particle is an M13 filamentous phage.

In embodiments, the first polymer is a cationic polymer. In embodiments, the first polymer is an anionic polymer. Where the charged coat protein is electrostatically bound to a first polymer, the charged coat protein and the first polymer are connected through an ionic bond.

In embodiments, the first polymer is or includes a polypeptide. In embodiments, the polypeptide has a net positive charge. In embodiments, the polypeptide encompasses a polymer of lysine (aka oligolysine, e.g., 3 to 20 lysine, 4 to 19 lysine, 5 to 18 lysine, 6 to 17 lysine, 7 to 17 lysine, 8 to 16 lysine, 9 to 16 lysine, 10 to 15 lysine, 11 to 14 lysine, 12 to 14 lysine, 13 to 14 lysine, 14 lysine). In embodiments, the polypeptide includes a polymer of lysine. In embodiments, the polymer of lysine is K₂, K₃, K₄, K₅, K₆, K₇, K₈, K₉, K₁₀, K₁₁, K₁₂, K₁₃, K₁₄, K₁₅, K₁₆, K₁₇, K₁₈, K₁₉, or K₂₀ (SEQ ID NO:4). The term “AAₓ” refers in the usual and customary sense to a polymer of amino acid “AA” having “x” repeating amino acid units. Thus, “K₂” refers to a lysine-lysine polymer or polymer portion (e.g. KK), “K₃” a lysine-lysine-lysine polymer or polymer portion (e.g. KKK), and so forth. In embodiments, the polymer of lysine includes 2 lysine residues (K₂), 3 lysine residues (K₃), 4 lysine residues (K₄), 5 lysine residues (K₅), 6 lysine residues (K₆), 7 lysine residues (K₇), 8 lysine residues (K₈), 9 lysine residues (K₉), 10 lysine residues (K₁₀), 11 lysine residues (K₁₁), 12 lysine residues (K₁₂), 13 lysine residues (K₁₃), 14 lysine residues (K₁₄), 15 lysine residues (K₁₅), 16 lysine residues (K₁₆), 17 lysine residues (K₁₇), 18 lysine residues (K₁₈), 19 lysine residues (K₁₉) or 20 lysine residues (K₂₀). In embodiments, the polymer of lysine includes 14 lysine residues (K₁₄).

In embodiments, the recognition moiety or ligand is a composition (e.g., atom, molecule, ion, molecular ion, compound, particle, protein, peptide, nucleic acid, oligosaccharide, polysaccharide, small molecule) capable of binding (e.g. specifically binding) to another complementary composition (e.g., analyte, polymer, protein, marker, small molecule, ligand, polysaccharide, aptamer, or other binder) to form a complex. In embodiments, a recognition moiety as provided herein may without limitation bind to biomolecules (e.g., hormones, cytokines, proteins, nucleic acids, lipids, carbohydrates, cellular membrane antigens and receptors (neural, hormonal, nutrient, and cell surface receptors or their ligands)); whole cells or lysates thereof (e.g., prokaryotic (e.g., pathogenic bacteria), eukaryotic cells (e.g., mammalian tumor cells); viruses (e.g., retroviruses, herpesviruses, adenoviruses, lentiviruses and spores); chemicals (e.g., solvents, polymers, organic materials, small molecules); therapeutic molecules (e.g., therapeutic drugs, abused drugs, antibiotics); environmental pollutants (e.g., pesticides, insecticides, toxins).

In embodiments, the recognition moiety or ligand is a cell surface marker binding moiety. In embodiments, the recognition moiety is a cancer cell surface marker binding moiety. In embodiments, the recognition moiety is a prostate-specific membrane antigen (PMSA) binding moiety.

The term “cancer cell marker” as used herein, refers to a protein or a polypeptide derived from a cancer cell or tumor that can be used to identify the cancer cell. A number of cancer cell markers have been established. The recognition moiety of the viral composition described herein can be designed to bind any cancer cell marker that is located on the surface of the cancer cell (i.e., any cancer cell surface marker) known in the art. Exemplary cancer cell markers that can be recognized and bound by the recognition moiety of the viral composition described herein include, but are not limited to,
(a) where the tumor cell is a breast cancer cell, the antigen may be one of EpCAM (epithelial cell adhesion molecule), Her2/neu (Human Epidermal growth factor Receptor 2), MUC-1, EGFR (epidermal growth factor receptor), TAG-12 (tumor associated glycoprotein 12), IGF1R (insulin-like growth factor 1 receptor), TACSTD2 (tumor associated calcium signal transducer 2), CD318, CD340, CD104, or N-cadherin;
(b) where the tumor cell is a prostate cancer cell, the antigen may be one of EpCAM, MUC-1, EGFR, PSMA (prostate specific membrane antigen), PSA (prostate specific antigen), TACSTD2, PSCA (prostate stem cell antigen), PCSA (prostate cell surface antigen), CD318, CD104, or N-cadherin;
(c) where the tumor cell is a colorectal cancer cell, the antigen may be one of EpCAM, CD66c, CD66e, CEA (carcinoembryonic antigen), TACSTD2, CK20 (cytokeratin 20), CD104, MUC-1, CD318, or N-cadherin;
(d) where the tumor cell is a lung cancer cell the antigen may be one or CK18, CK19, CEA, EGFR, TACSTD2, CD318, CD104, or EpCAM;
(e) where the tumor cell is a pancreatic cancer cell the antigen may be one of HSP70, mHSP70, MUC-1, TACSTD2, CEA, CD104, CD318, N-cadherin, or EpCAM1;
(f) where the tumor cell is an ovarian cancer cell the antigen may be one of MUC-1, TACSTD2, CD318, CD104, N-cadherin, or EpCAM;
(g) where the tumor cell is a bladder cancer cell, the antigen may be one of CD34, CD146, CD62, CD105, CD106, VEGF receptor (vascular endothelial growth factor receptor), MUC-1, TACSTD2, EpCAM, CD318, EGFR, 6B5 or Folate binding receptor;
(h) where the tumor cell is a cancer stem cell, the antigen may be one of CD133, CD135, CD 117, or CD34; and
(i) where the tumor cell is a melanoma cancer cell, the antigen may be one of the melanocyte differentiation antigens, oncofetal antigens, tumor specific antigens, SEREX antigens or a combination thereof. Examples of melanocyte differentiation antigens, include but are not limited to tyrosinase, gp75, gp100, MART 1 or TRP-2. Examples of oncofetal antigens include antigens in the MAGE family (MAGE-A1, MAGE-A4), BAGE family, GAGE family or NY-ESO1. Examples of tumor-specific antigens include CDK4 and 13-catenin. Examples of SEREX antigens include D-1 and SSX-2.

In embodiments, the recognition moiety is a polypeptide. In embodiments, the recognition moiety is a peptide having the sequence of CALCEFLG (SEQ ID NO:5). In embodiments, the recognition moiety is a peptide having the sequence of SECVEVFQNSCDW (SEQ ID NO:6). In embodiments, the recognition moiety is a polypeptide that is selected through a phage display library and this polypeptide selectively recognizes and binds to a cell surface marker. In embodiments, the recognition moiety encompasses an antibody, a variant or a fragment thereof, where the antibody (a variant or a fragment thereof) specifically recognizes and binds to a surface marker on a cell (e.g., a cancer cell).

In embodiments, cell is a target cell. The term “target cell” and the like refer, in the usual and customary sense, to a cell which can indicate a pathological condition or the potential for a pathological condition, e.g., a disease. In embodiments, the target cell expresses a surface marker for a disease, as disclosed herein. In embodiments, the target cell is a non-pathological cell, e.g., a normal cell, the identification of which is desired, e.g., within a biological sample.

In embodiments, the linker is attached to one end of the main backbone of the recognition moiety (e.g., —COOH group at the C-terminus of a peptide recognition moiety). In embodiments, the linker is attached to a side chain of the recognition moiety (e.g., a —COOH group of a side chain of a peptide recognition moiety).

In another aspect, there is provided a complex including a viral composition as disclosed herein and a phospholipid vesicle. In embodiments, the phospholipid vesicle is a cell. It is well understood that living cells include a membrane structure defining the surface of the cell, which membrane structure includes a phospholipid bilayer. Within the phospholipid bilayer can be found a variety of biomolecules including proteins, lipids, small molecules (e.g., cholesterol and esters thereof), attachment points for cytoskeletal structures, and other chemical structures which provide recognition signals for the cell. In embodiments, proteins attached to the cellular membrane provide a charged protein coat for the cell or vesicle. In embodiments, the phospholipid vesicle is a cell-derived vesicle. In embodiments, the phospholipid vesicle is an exosome. The term “exosome” and the like refer, in the usual and customary sense, to a cell-derived vesicle that is present in many biological fluids, typically having a size on the order to 30 to 100 nm, which can be released from the cell. Without wishing to be bound by any theory, it is believed that exosomes function in intercellular signaling, cellular functioning (e.g., coagulation), waste management, and other cellular functions. Exosomes can contain molecular constituents including proteins (e.g., recognition proteins), and nucleic acid (e.g., RNA). Indeed, exosomes are implicated in cellular transfer reactions from one cell to another via membrane vesicle trafficking, as known in the art, thereby providing pathways for intercellular communication in e.g., the immune system.

Also provided herein is a complex that includes any viral composition described herein and a cell, where the recognition moiety of the viral composition is bound to the cell. In embodiments, the cell is a cancer cell. In embodiments, the cancer cell has a cancer cell surface marker (i.e., a tumor cell antigen) to which the recognition moiety of the conjugated wrapped phage binds.

In embodiments, the complex includes one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more) viral compositions described herein, where the recognition moiety of each viral composition is bound to a cell (e.g., a cancer cell). In embodiments, the recognition moiety of each viral composition is the same. In embodiments, the recognition moiety of each viral composition is not the same. In embodiments, the recognition moiety of each viral composition is a same cell surface marker binding moiety. In embodiments, the recognition moiety of each viral composition is not a same cell surface marker binding moiety. In embodiments, the recognition moieties of the 2 or more viral compositions are not the same cell surface marker binding moieties, but they recognize and bind to the same cell surface marker. For example, they recognize and bind to different sites of the same cell surface marker. In embodiments, the recognition moieties of the 2 or more viral compositions recognize and bind to 2 or more different cell surface markers (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more cell surface markers) of a cell (e.g., cancer cell).

Also provided is a pharmaceutical composition that includes any viral composition described herein and a pharmaceutically acceptable carrier, diluent or excipient.

In embodiments, the pharmaceutical composition includes one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more) viral compositions described herein, where the recognition moiety of each viral composition is capable of binding to a cell surface marker (e.g., a cancer cell surface marker). In embodiments, the recognition moiety of each viral composition is the same. In embodiments, the recognition moiety of each viral composition is not the same. In embodiments, the recognition moiety of each viral composition is a same cell surface marker binding moiety. In embodiments, the recognition moiety of each viral composition is not a same cell surface marker binding moiety. In embodiments, the recognition moieties of the 2 or more viral compositions are not the same cell surface marker binding moieties, but they recognize and bind to the same cell surface marker. For example, they recognize and bind to different sites of the same cell surface marker. In embodiments, the recognition moieties of the 2 or more viral compositions recognize and bind to 2 or more different cell surface markers (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more cell surface markers) of a cell (e.g., cancer cell).

In embodiments, provided herein are articles of manufacture or kits containing any compositions described herein (e.g. viral particle, phage, phage wrappers or linkers, ligands or recognition moieties, first polymer) and instructions for their use in the methods described herein.

Methods

In another aspect, there is provided a method for detecting a cancer cell in a subject. The method includes (a) contacting a biological sample of the subject with one or more viral compositions described herein, where the recognition moiety of one or more viral compositions is a cancer cell surface marker binding moiety, and (b) detecting a cell-viral composition complex, and presence of the complex indicates presence of a cancer cell in the subject.

In another aspect, there is provided a method for diagnosing a cancer in a subject. The method includes (a) contacting a biological sample of the subject with one or more viral compositions described herein, where the recognition moiety of one or more viral compositions is a cancer cell surface marker binding moiety, and (b) detecting a cell-viral composition complex, and presence of the complex indicates presence of a cancer cell in the subject, thereby diagnosing a cancer in the subject.

In another aspect, there is provided a method for detecting aggressiveness of a disease in a subject. The method includes (a) contacting a biological sample of the subject with one or more viral compositions described herein, where the recognition moiety of one or more viral compositions is a cancer cell surface marker binding moiety, (b) detecting a cell-viral composition complex, and (c) quantitating the complex (total number or concentration of the complex), thereby detecting aggressiveness of the disease in the subject. In embodiments, the higher total number or concentration of the complex indicates a higher level of aggressiveness of the disease. In embodiments, the disease is a cancer.

In embodiments, the recognition moiety of each viral composition used in the method described herein is the same. In embodiments, the recognition moiety of each viral composition used in the method described herein is not the same. In embodiments, the recognition moiety of each viral composition is a same cell surface marker binding moiety. In embodiments, the recognition moiety of each viral composition is not a same cell surface marker binding moiety. In embodiments, the recognition moieties of the 2 or more viral compositions are not the same cell surface marker binding moieties, but they recognize and bind to the same cell surface marker. For example, they recognize and bind to different sites of the same cell surface marker. In embodiments, the recognition moieties of the 2 or more viral compositions recognize and bind to 2 or more different cell surface markers (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more cell surface markers) of a cell (e.g., cancer cell).

In embodiments, the molar ratio of different types of recognition moieties used in the compositions/methods described herein is optimized. In embodiments, the optimization leads to a synergistic binding between the recognition moieties and the cell surface marker. In embodiments, the synergistic binding between the recognition moieties and the cell surface marker results in higher sensitivity and/or higher specificity of the method described herein.

In embodiments, methods described herein utilize two types of viral compositions that each includes one unique recognition moiety. In embodiments, the molar ratio of these two types of viral compositions used in the methods described herein is optimized. In embodiments, the ratio of two recognition moieties or two types of viral compositions is, for example, 1:1, 1:2, 1:3, 1:4, 1:5, 1:6, 1:7, 1:8, 1:9, 1:10, 10:1, 9:1, 8:1, 7:1, 6:1, 5:1, 4:1, 3:1, 2:1.

In embodiments, the one or more viral compositions used in the methods described herein are immobilized to a solid support.

Methods for detecting a cell-viral composition complex are known in the art. In embodiments, the detecting includes an antibody based reaction. In embodiments, the binding of a viral composition to a cell (i.e., the binding of a recognition moiety to a cell surface marker) can be detected and/or quantified using a variety of techniques such as, but not limited to, Western blot, dot blot, surface plasmon resonance method (e.g., BIAcore system; Pharmacia Biosensor AB, Uppsala, Sweden and Piscataway, N.J.), isothermal titration calorimetry (ITC), or enzyme-linked immunosorbent assays (ELISA).

Immunoassays which can be used to analyze immunospecific binding and cross-reactivity of the recognition moiety include, but are not limited to, competitive and non-competitive assay systems using techniques such as Western blots, RIA, ELISA (enzyme linked immunosorbent assay), “sandwich” immunoassays, immunoprecipitation assays, immunodiffusion assays, agglutination assays, complement-fixation assays, immunoradiometric assays, and fluorescent immunoassays. Such assays are routine and well known in the art.

In another aspect, there is provided a method for generating a viral composition described herein. The method includes (a) synthesizing a covalent linker comprising -L¹-L²-L³-L⁴-L⁵-L⁶-, where L¹, L², L³, L⁴, L⁵ and L⁶ are independently a bond, —O—, —C(O)O—, —C(O)—, —C(O)NH—, —NH—, —S—, —S(O)2NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl; (b) contacting the linker with a recognition moiety thereby forming an intermediate linker-recognition moiety conjugate; and (c) contacting the intermediate linker-recognition moiety conjugate with an intermediate wrapped phage thereby forming said viral composition, and the intermediate wrapped phage includes a whole viral particle having a charged protein coat that encompasses a charged coat protein and a first polymer electrostatically bound to the charged coat protein.

Exemplary reagents and steps for generating a viral composition described herein are provided in the figures (e.g., FIG. 15) and Examples below.

### EXAMPLES

### Example 1

### Design and Rationale and Protocols

There are provided methods and compositions for the selective detection of circulating tumor cells. M13 bacteriophage (termed ‘phage’) are used as the scaffold for the selective capture of target biomarker. But, phage typically adheres to cell surfaces with high affinity, which presents a huge problem. A similar problem arises with phage-based sensors for the detection of tumor cells; the non-specific background can reduce signal to noise, and the ability to distinguish tumor from non-tumor cells.

Phage typically adheres to cell surfaces with high affinity. Such non-specific adhesion complicates the design of phage-based sensors for the detection of tumor cells; the non-specific background can reduce the signal to noise ratios and the ability to distinguish tumor from non-tumor cells. The approach described here, applies non-covalent attachment to the phage surface to access additional architectures, e.g., for biosensor applications. Non-covalent attachment offers comparable stability to covalent modification of the virus surface; for technical reasons, non-covalent attachment offers easier execution than covalent bond formation to the surface of the virus. The high negative charge on the phage surface allows non-covalent wrapping with cationic peptides and polymers. Linking these wrappers to recognition ligands opens new routes to greater sensitivity and specificity for target analytes. The peptide ligands can be chemically synthesized and fused to an oligolysine peptide (K₁₄), which ‘wraps’ around the virus particle through complementary electrostatic interactions. Previously, this strategy allowed maximization of ligand density on the phage surface for sensitive detection of biomarkers in complex biofluids, such as synthetic urine. Here, the overall design incorporates PEG polymers in conjunction with this wrapping strategy to address the problem of non-specific adhesion between phage and cells. Then, we optimize various architectures for the specific detection of PCa cells.

Among prostate cancer cell lines, LNCaP cells provide the most commonly used in vitro model for early stage PCa. Derived from the lymph node adenocarcinoma of the human prostate, LNCaP expresses most of the important PCa biomarkers including Prostate Specific Membrane Antigen (PSMA), Prostate Specific Antigen (PSA) and Androgen Receptor (AR). Attempts to recognize cell surfaces with conventional phage-displayed ligands resulted in unacceptably high, non-specific adhesion by control phage, which lack a displayed peptide. As shown by ELISA, phage-displayed PSMA ligand 2 and control phage produced similar high levels of binding to LNCaP cells. To overcome this non-specific adhesion, we focused on eliminating such interactions by control phage by using polyethylene glycol.

The water-soluble polymer PEG is commonly bioconjugated to proteins to reduce non-specific adhesion to cells and other surfaces. PEG has been shown to broadly adopt two distinct conformations—descriptively termed ‘mushroom’ and ‘brush.’ The transition from the mushroom conformation, a more random orientation, to the brush conformation is dependent upon the polymer length and packing densities; longer PEG lengths and higher packing densities favor formation of the brush conformation. This transition can result in a significant drop in non-specific adsorption. In many systems, a mole fraction of 0.15 PEG-modified to -unmodified sites yields significantly reduced non-specific adhesion. High packing densities with such mole fractions can force the polymer to adopt a more stretched, and extended brush conformation to more effectively suppress non-specific adhesion.

Initial attempts to block non-specific cell adhesion applied PEG variants with different MWs as phage wrappers. Azide-functionalized, polydispersed PEGs with size distributions centered around 7, 22 or 45 ethylene glycol units (providing average MWs of 300, 1K or 2K, respectively) were conjugated to K₁₄-alkyne using the Cu^{I}-catalyzed cycloaddition (‘click’) reaction. The conjugated peptides were then purified by reverse-phase HPLC and characterized by MALDI-TOF mass spectrometry. The relative adhesion levels of unwrapped and PEG-wrapped phage targeting immobilized LNCaP cells were compared by phage-based ELISA. Since the phage lacked a displayed peptide, adhesion could only result from non-specific interactions by the phage coat proteins. In theory, phage wrapped with PEG should bind to LNCaP cells with much lower affinity due to decreased non-specific adhesion. However, no such reduction was observed for the different MW PEGs used. The ineffectiveness of this initial approach likely resulted from interaction between PEG and the K₁₄ sidechains used to wrap the phage. A crown ether-like encapsulation can form between the primary amine of the Lys sidechains and ethylene glycols of PEG, thereby could render K₁₄ incapable of wrapping the phage surface. Without the PEG wrapping the phage surface, the results merely compare phage in different assay wells, as is evident from the overlapping responses. The lack of wrapping by PEGylated oligolysine was further verified by dynamic light scattering measurements, which revealed no significant change in the cross-sectional diameter of the treated phage.

On-Phage Cycloaddition Reaction to Generate PEGylated Phage.

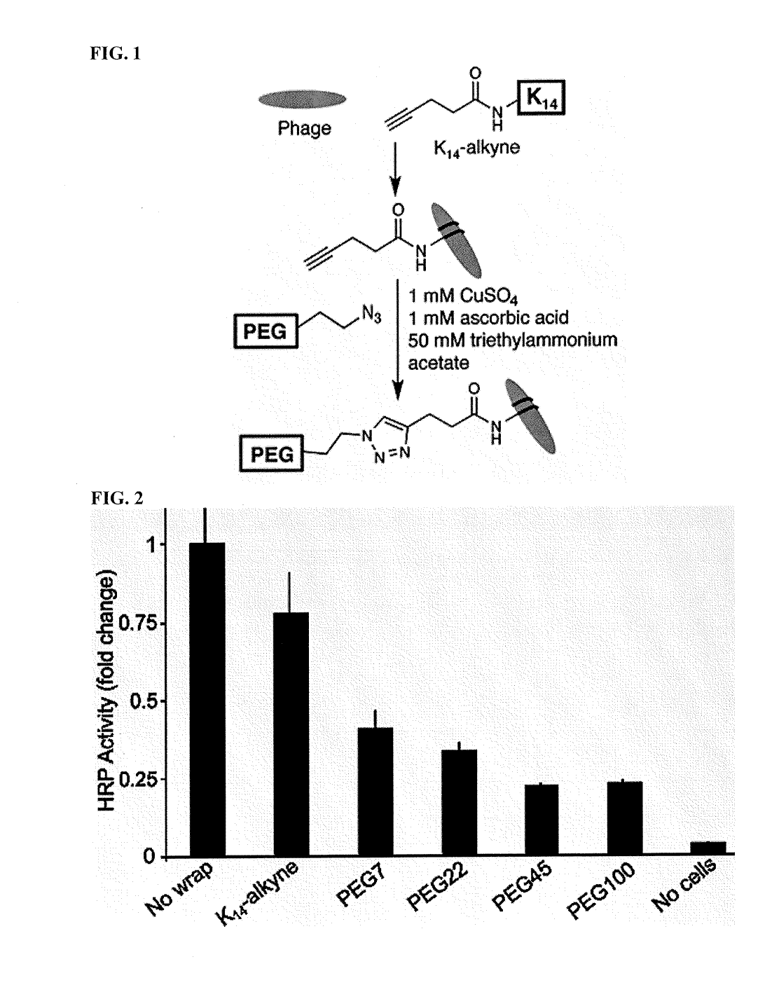

To overcome K₁₄ encapsulation by PEG, phage with PEG wrappers were generated in two steps, FIG. 1. First, phage were wrapped with K₁₄-alkyne by incubation at room temperature for 15 min. During this step, the K₁₄-alkyne wrap the phage prior to PEG conjugation. Next, PEG azides were added, and the cycloaddition reaction with the K₁₄-alkyne took place on the phage surface for 30 min. Next, the ELISA described above was repeated with these PEGylated phage comparing the non-specific adhesion of phage wrapped with different MW PEGs, FIG. 2; in this experiment negative controls of ‘No wrap’ and ‘No cells’ indicate the extremes of high levels of non-specific adhesion to LNCaP cells and non-binding, respectively. Phage wrapped with PEG45 and PEG100 (average MW 2K and 5K, respectively) demonstrate a >75% reduction in non-specific binding to LNCaP cells, as demonstrated by the observed decrease in HRP activity resulting from lower phage binding. The experiment confirms that PEG wrappers can effectively suppress non-specific adhesion, provided the K₁₄ wraps around the phage first. The reduction in non-specificity increases with larger MW PEG polymers, and saturates at around 45 ethylene glycol units.

PEG reduces non-specific binding largely by surrounding the attached surface with a hydration sphere. The non-specific binding partners could adhere to the outer surface of the PEG layer, termed secondary adsorption. For non-specific adhesion to the larger surfaces of cells, such secondary adsorption is likely a more pronounced effect.

Dynamic Light Scattering (DLS) Analysis of PEGylation.

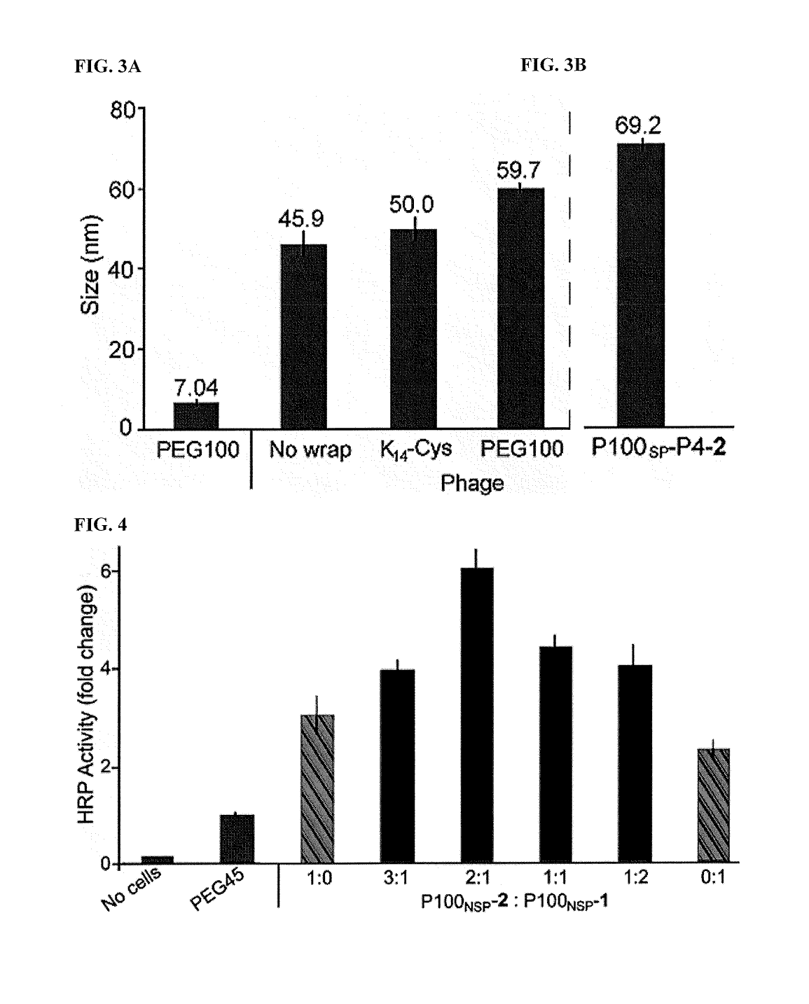

To characterize the PEG-wrapped phage, DLS measurements were conducted, FIGS. 3A-3B. Next, the change in average cross-sectional diameter was measured for different samples from each step of the phage wrapping process, FIG. 3A. The addition of K₁₄-Cys wrappers on the phage leads to an increase in cross-sectional diameter from 45.9 to 50.0 nm. Upon conjugation of this K₁₄-Cys wrapped phage to maleimide-functionalized PEG100, an approximate 10 nm increase in size is observed. This increase in size matches two independent reports for size increases after PEG100 bioconjugation to gold nanoparticles. Thus, the DLS-based size measurements confirm the formation of the expected phage-wrapped complexes.

Synthesis of PEGylated Ligands.

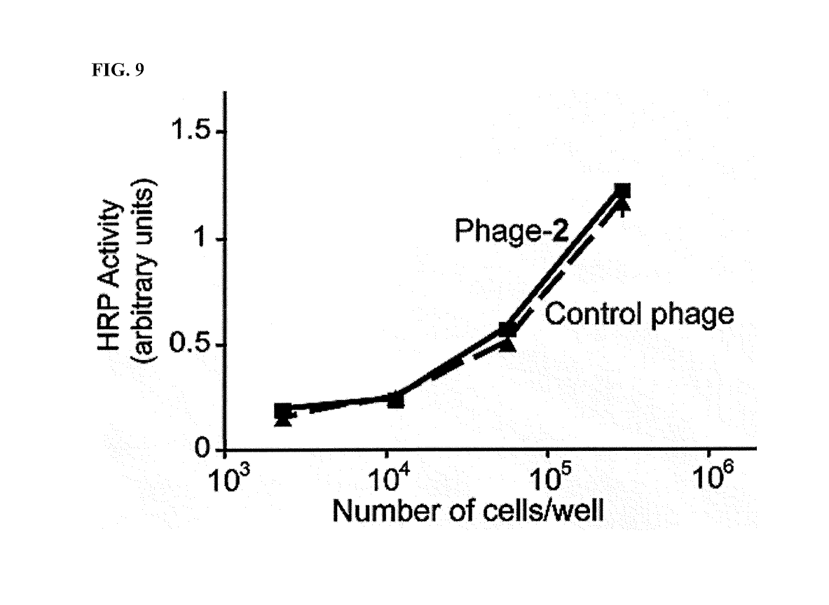

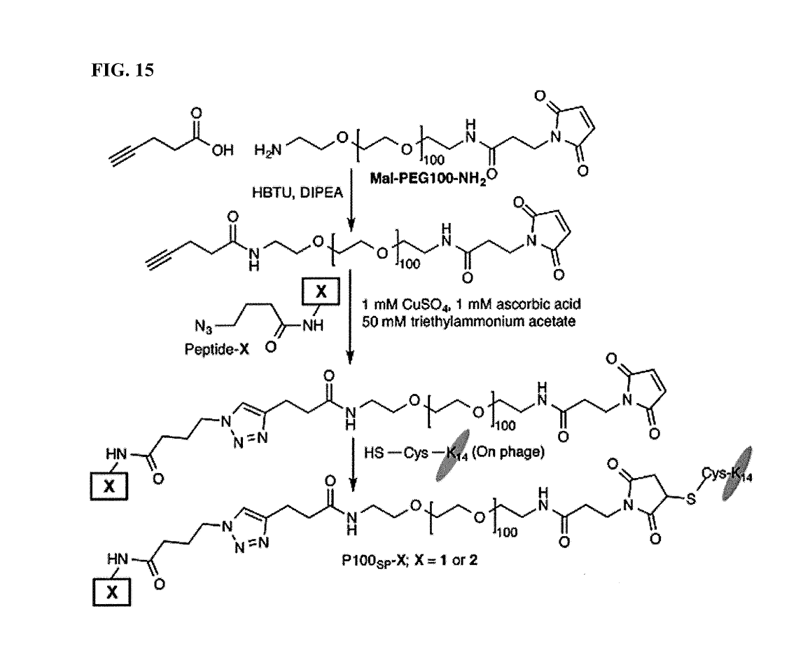

Towards the goal of specific recognition of a cell surface receptor, different scaffolds for the display of ligands on phage were explored. First, heterobifunctional PEG, Mal-PEG-NH₂, provided reactive groups for selective attachment of oligolysine at the maleimide end and PSMA binding ligands to the amine end, Scheme 1. As the scaffold, PEG100 was chosen to provide a longer polymer brush to reduce non-specific secondary adsorption. Azide-functionalized PSMA ligands 1 and 2 were synthesized by conventional solid-phase peptide synthesis (SPPS), and coupled to pentynoic acid via the click reaction. The resultant N-terminal carboxylic acid group was then coupled to Mal-PEG100-NH₂ using HBTU as an amide bond forming agent in water. Since this reaction non-specifically couples amine and carboxylate functionalities, the attachment sites could vary as both ligands have sidechain carboxylic acids. The resultant ligand is termed P100_{NSP}-1/2 for ‘PEG100, non-specific attachment to ligand 1 or 2.’ as described in Table 1. Subsequently, phage wrapped with K₁₄-Cys were coupled to the maleimide terminus of P100_{NSP}-1/2, as described above. Preliminary validation of binding to cell surface PSMA by the PEGylated ligand wrappers was performed by phage ELISA as before, FIG. 14. Compared to the non-specific binding observed in FIG. 9, a slight improvement in binding affinity resulted from wrapping with the PEGylated ligands. This modest result provided a starting point for further engineering. Phage wrapped with P100_{NSP}-2 displayed a higher affinity for LNCaP cells compared to P100_{NSP}-1 as expected from its higher binding affinity for PSMA.

Bidentate Binding Mode of PEGylated Ligands.

Dual display of ligands 1 and 2 can enable synergistic, high affinity binding to PSMA due to a bidentate binding mode and a hook and loop-like avidity effect. A phage ELISA targeting LNCaP cells with different ratios of the two PEGylated, phage-wrapped ligands examined relative binding affinities. First, the effectiveness of the bidentate binding mode (FIG. 4) was compared to binding by individual ligands (patterned bars in FIG. 4). Having two ligands on the phage surface consistently improved binding affinity. Furthermore, a 2:1 mixture of P100_{NSP}-2 and P100_{NSP}-1 respectively, was found to maximize the recognition of PSMA on LNCaP cells. The improved binding from a 2:1 ratio stems from the higher binding affinity of ligand 2 compared to ligand 1. Altering this ratio in either direction drops the apparent affinity, likely due to loss of optimal bidentate binding. Hereafter, phage were wrapped with a 2:1 mixture of the PEGylated ligands 2 and 1, respectively.

Optimizing the Attachment Site for PEGylated Ligands.

Further optimization explored the size, geometry and attachment site of the ligands fused to the PEG wrapper. Such variables can be crucial to the pharmacokinetic properties of PEGylated drugs, which demonstrates the sensitivity of biological recognition to such factors. For example, the attachment sites of the peptide ligand to PEG100 dictates the ligand orientation and the potential availability of peptide sidechains. An alternative synthesis scheme was designed to control ligand orientation. Mal-PEG100-NH₂ was first coupled to pentynoic acid. The resultant Mal-PEG100-alkyne was then coupled to the azide-functionalized peptide ligands using click chemistry, providing a specific site of attachment to the ligand. The resulting PEGylated ligand is termed P100_{SP}-1/2 for ‘PEG100, specific attachment to ligand 1 or 2,’ as described in Table 1.

Insertion of a PEG4 linker to reduce steric effects on the attached ligands. Heterobifunctional linkers between PEG and a molecule of interest can enhance activity through flexible additional spacing. We envisioned the incorporation of an average 175 MW PEG4 linker between the peptide ligand and the triazole generated by the click reaction could enhance the binding affinity of the peptide ligands. With only four ethylene glycol units, this highly flexible linker can disconnect the peptide ligand from any steric constraints dictated by PEG100 or the triazole. Thus, the peptide ligands were resynthesized via SPPS, and coupled to azido-PEG4-carboxylic acid (15-Azido-4,7,10,13-tetraoxapentadecanoic acid), thereby inserting a PEG4 linker before the azide functionality. Azido-PEG4-ligands were further linked to PEG100 following the two synthesis routes described above, specific and non-specific addition. The resultant PEGylated ligands are termed P100_{SP}-P4-1/2 and P100_{NSP}-P4-1/2, P4 to indicate the insertion of the PEG4 linker, Table 1. The P100_{SP}-P4-1 and P100_{SP}-P4-2 conjugated peptides were shown to have the expected sizes by gel permeation chromatography (Supplementary Information) and DLS. A further increase of 10 nm in cross-sectional diameter was observed for the addition of the PEG4-fused ligand (FIG. 3B).

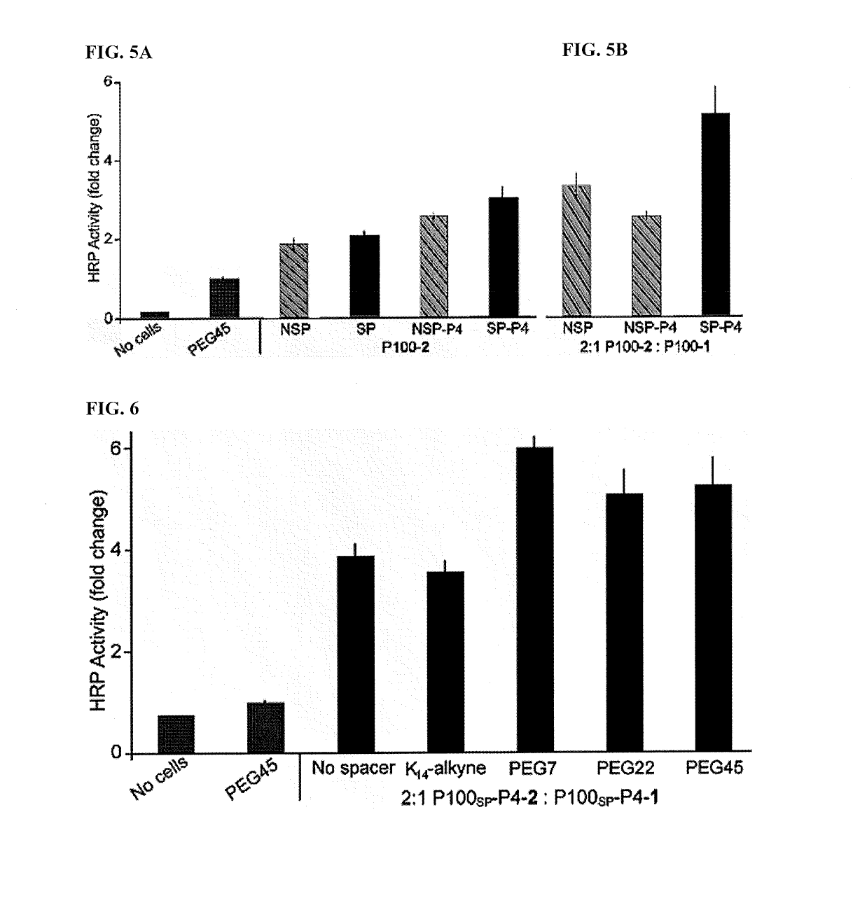

An ELISA compared the relative binding affinities of the four PEGylated ligand 2 variants—specific (solid) and non-specific (patterned) attachment with and without the PEG4 linker, FIG. 5A. P100_{SP}-2 demonstrates a higher binding affinity for cell surface PSMA than P100_{NSP}-2, illustrating the significance of the unmodified Glu sidechain obtained through specific attachment. Furthermore, inclusion of the PEG4 linker further enhances the binding affinity for both P100_{SP}-2 and P100_{NSP}-2. As a result, the PEGylated ligand P100_{SP}-P4-2 incorporating the PEG4 linker with specific attachment site provided the most effective architecture for the PEGylated ligand to recognize PSMA on the cell surface.

PEG Spacers to Control Relative Ligand Spacing.

The relative spacing between ligands governs the synergy of the chelate-based avidity effect. To achieve optimal geometry of the two ligands, the relative spacing was systematically engineered by interspersing long PEGylated ligands with smaller PEG wrappers on the phage surface. The smaller PEG wrappers could provide spacers to push apart the PEG-fused ligands on the phage surface. Generating ligands and spacers required the two wrapping modes described above, click chemistry and cysteine-maleimide reaction, on the same phage. K₁₄-alkyne and K₁₄-Cys were pre-mixed to an estimated mole fraction of 0.19, and then used to wrap the phage surface. K₁₄-alkyne was linked to short PEG polymers to provide spacers. Different concentrations of the PEG polymers were explored. The ratio of ligands to spacers was empirically optimized, and a ratio of 1.5:1 provided the best levels of PSMA recognition. The concentration of the PEGylated ligands remained unchanged, and a 2:1 molar mixture of the two ligands was reacted with the K₁₄-Cys wrapped on the phage surface. A higher net concentration of wrappers could be accommodated by the phage as the spacers allowed higher packing density. The dual PSMA ligand combination described above, P100_{SP}-P4-2+P100_{SP}-P4-1, without (green) or with spacers (brown) of either PEG 7, 22 or 45, wrapped around the phage were assayed for binding to LNCaP cells, FIG. 6. All spacers significantly enhanced PSMA recognition by the displayed ligands. However, the PEG7 spacer proved most effective. The much smaller PEG7 spacer can force the ligands into adopting a more optimal geometry for effective bidentate binding, and the height of this polymer brush does not interfere with ligand binding. Longer spacers failed to boost binding affinity to the same levels. At the mole fraction of PEG used, the PEG polymers can adopt the brush conformation with the height of the polymer brush dependent on the PEG length. Interdigitation of PEG spacers with PEGylated ligands can interfere with the binding affinity of the ligands, as shown with the longer brushes of PEG22 and 45. Also, the addition of K₁₄-alkyne without conjugated PEG spacers has no effect on binding affinity, as expected; thus, the increased packing of oligolysine wrappers is not a contributing factor. Rather, enhanced binding results from the improved geometry through addition of PEG spacers.

Selective Recognition of PSMA Positive Cells.

To demonstrate specificity for PCa cells by these chemically modified phage, binding to different prostate cancer cell lines was compared. LNCaP cells can model early or late stage cancer cells, through variation in their culture conditions. The majority of PCa cases gain resistance to therapies based on androgen ablation. The LNCaP cell line, a model for early stage PCa, is androgen sensitive but gradually loses the androgen requirement, providing a model for late stage PCa, which also mimics androgen ablation. The latter can be simulated by culturing LNCaP cells in androgen-depleted media, referred to as LNCaP CSS (for charcoal-stripped serum). Increased levels of PSMA are associated with androgen independent PCa. Thus, both LNCaP and LNCaP CSS cell lines were assayed. The third cell line, PC3 cells, do not express PSMA, and were used as the negative control. The following assays validate the dual ligand system for cell line discrimination and quantification of cell surface receptors.

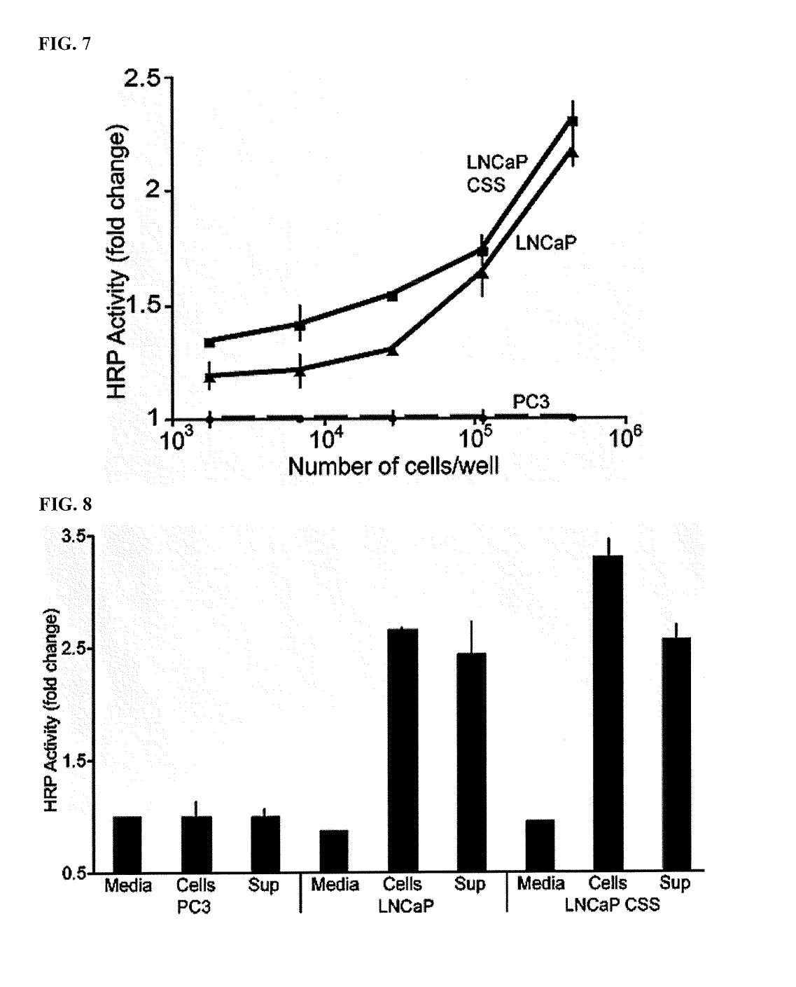

The optimized dual ligand combination of P100_{SP}-P4-2+P100_{SP}-P4-1 and the PEG7 spacer was assayed for binding to LNCaP (red), LNCaP CSS (blue), and PC3 cell lines (gold), FIG. 7. The results demonstrate high specificity for PSMA positive LNCaP cells in a dose-dependent manner with higher apparent affinity to LNCaP CSS cells. This higher sensitivity to LNCaP CSS cells is consistent with the increase in PSMA expression resulting from the progression of the cancer cells to an androgen independent state in the LNCaP CSS model.

Detecting PSMA on Suspended Cells and in Culture Media.

The tailored phage could also capture cells from solution, which is critical for future analytical applications in circulating tumor cell detection and characterization. In this experiment unlike other ELISAs described here, the phage were immobilized on the microtiter plate before applying a solution of cells, FIG. 8. Again, phage wrapped with the dual ligand combination of P100_{SP}-P4-2+P100_{SP}-P4-1 and the PEG7 spacer were used. In this experiment, the capture of PSMA positive cells is detected by and proportional to cell surface PSMA concentration. PC3 cells, lacking PSMA, do not generate a significant response, as expected. PSMA levels are elevated in the urine samples of PCa patients, and levels of this biomarker correlate with the aggressiveness of the disease. Therefore, cultured PCa cells should release PSMA into their culture media. Thus, PSMA detection was also performed with cell culture supernatant, normalized to the volume and the number of cells (FIG. 8). The PEGylated dual ligand combination on phage allows sensitive PSMA detection in 100 μL of supernatant from both LNCaP and LNCaP CSS cell cultures. Cell culture media from PC3 cells and fresh culture medias serve as the negative controls. As expected, the negative controls failed to show any significant binding. The effective detection of PSMA shed by LNCaP cells, in androgen sensitive and androgen independent cells, demonstrates the use of phage wrapped with PEGylated ligands for future development of analytical devices and translation to the clinic.

Protocols

Synthesis of PEGylated Ligands—Specific Attachment Mode.

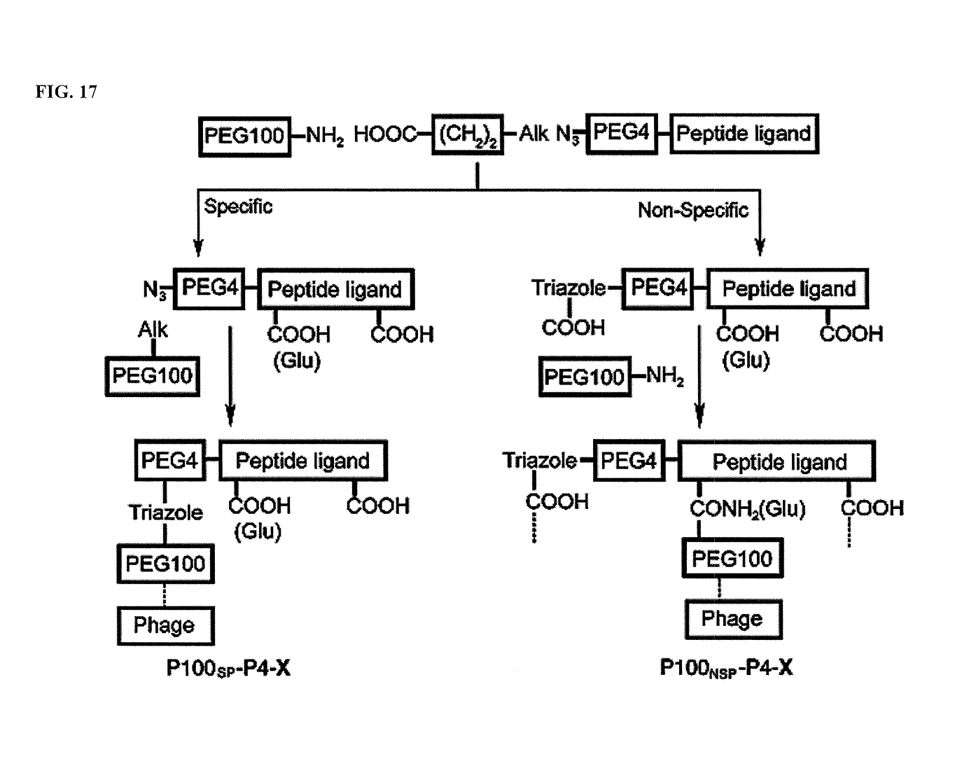

The protocol for the synthesis of the PEGylated ligands was adapted from the solid phase peptide synthesis and click chemistry reaction described above, and also described in FIGS. 15 and 17. In a glass test tube, 40 μL of 1 mM Mal-PEG100-NH₂ (commercially purchased from Alfa Aesar) in water, 12 μL of 10 mM pentynoic acid (in water), 12 μL of 10 mM HBTU (in NMP), 40 μL of DIPEA and 296 μL of HPLC grade water were combined and stirred at room temperature for 2 h, yielding alkyne-functionalized-PEG100-Mal. To obtain more quantities of the product, multiple reactions at this volume were run in parallel. To remove unreacted starting material and to concentrate the product, the reaction mixtures were diluted with an equal volume HPLC grade water before concentration to ⅕^{th} volume using 2K MWCO concentrators (Sartorius).

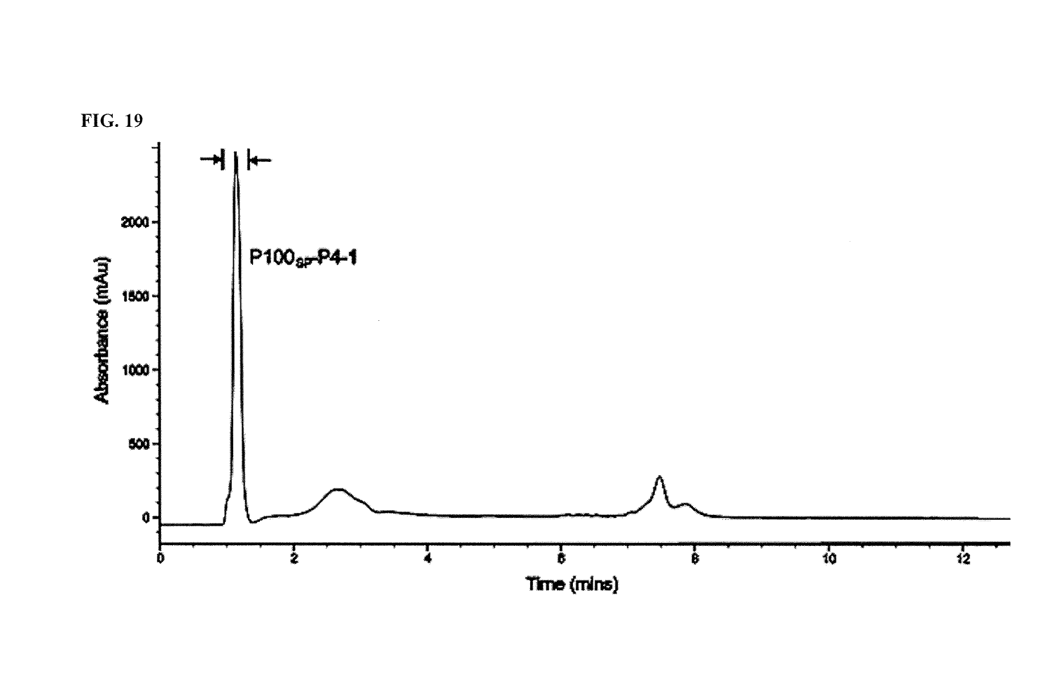

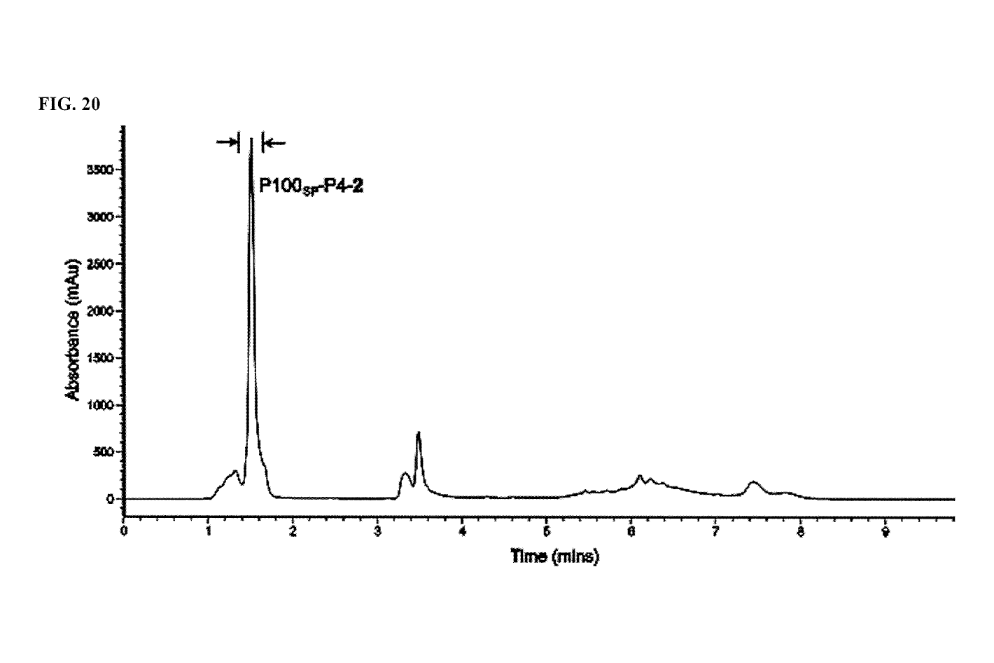

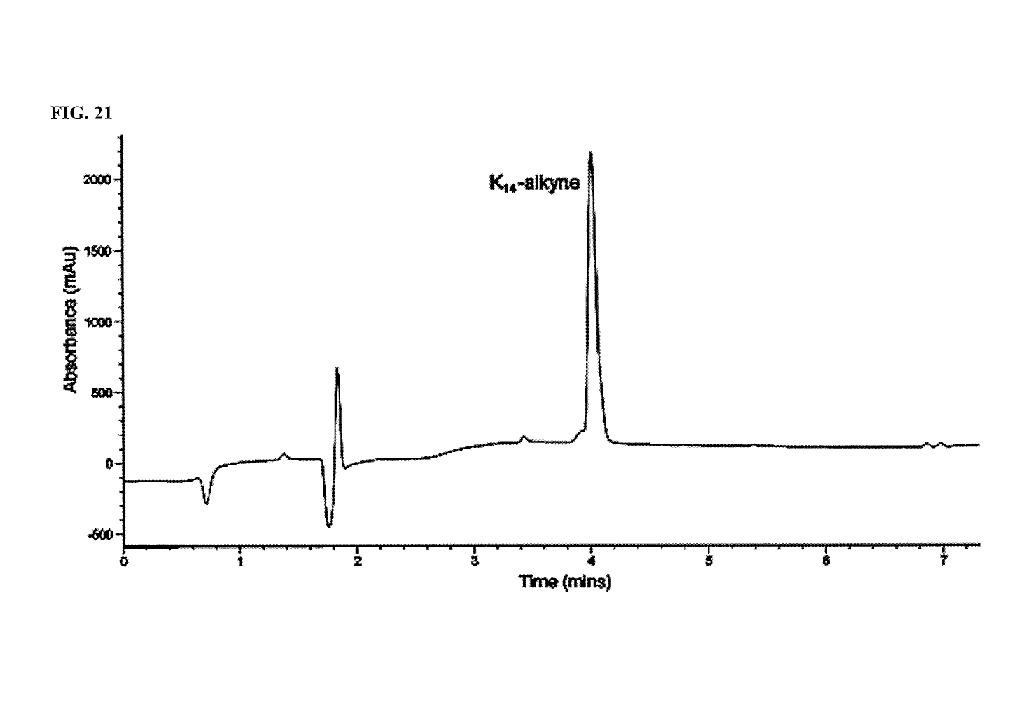

For the next step of the synthesis, ˜50 μL of the alkyne-functionalized-PEG100-Mal, obtained as described above, was conjugated to azide-functionalized peptide ligands (final concentration of 40 μM) by click chemistry as before [1,5]. The reaction mixture was stirred overnight at room temperature. Four identical reactions were run in parallel. To remove unreacted starting materials, the four reaction mixtures were then combined and concentrated to ¼^{th} volume using 3K MWCO concentrators. Next, the resultant solution was diluted with an equal volume of HPLC grade water before concentrating to ˜½ volume using 5K MWCO concentrators. The concentrated reaction mixture was purified using reverse-phase analytical HPLC (FIGS. 19-20) and fractions were identified by MALDI-TOF mass spectrometry. The high polydispersity of the high MW PEG polymer prevents accurate mass determination by this technique, and instead Gel Permeation Chromatography (GPC) was used as described in the next section.

For the non-specific attachment mode described in the text and FIG. 17, the PEGylated ligands were synthesized in the reverse order. The azide-functionalized peptides were first conjugated to pentynoic acid using click chemistry. The resultant peptide was then coupled to Mal-PEG100-NH₂ using HBTU and DIPEA as described above.

Cell Growth.

The cell lines were grown as monolayers in media supplemented with 10% fetal bovine serum (Cellgro), 1 mM sodium pyruvate [7] and 1% penicillin-streptomycin-glutamine in a 5% CO₂ and 95% air-humidified atmosphere at 37° C. LNCaP cells were cultured in RPMI 1640 media. For studies with LNCaP cells cultured in charcoal-stripped serum (LNCaP CSS cells), LNCaP cells were washed with PBS and then incubated with phenol free RPMI 1640 media supplemented with 10% charcoal-stripped serum (Cellgro) for five minutes. The cells were again washed with PBS, and provided fresh media [8]. PC3 cells were grown in Ham's F-12 media.

Cell-Based Phage Enzyme-Linked Immunosorbent Assay (ELISA).

Day 1: The cell-based ELISA was performed as previously described by Watanabe et. al. with the following modifications [9]. Cells were detached with Trypsin-EDTA, resuspended in PBS, and then collected by centrifuging at 1200 rpm for 5 min. The cells were further washed with PBS and then concentrated as in the previous step. The concentration of the cells was adjusted to 4.5×10⁶ cells/mL in PBS using a hemocytometer, and 100 μL was aliquoted to specific wells of a 96-well microtiter plate (Maxisorp plates from Nunc). The Maxisorp plates used here have a high protein-binding capacity. Thus, the plates can be used to run assays with either the cells or the phage immobilized in the wells. Next, 50 μL of a 0.15% glutaraldehyde in PBS solution was added to the wells at 4° C., and the solution was gently mixed by pipetting. The ELISA plate was then centrifuged at 1200 rpm for 10 min at 4° C., followed by overnight incubation at 4° C.

Day 2: The cell solution was gently removed, and the wells were blocked with 200 μL/well of blocking buffer containing 100 mM glycine, 1% gelatin and 0.1% w/v BSA (bovine serum albumin) in PBS. The plate was incubated overnight (˜20-22 h) at room temperature.

Separately, phage were prepared before attachment to PEG and PEGylated ligands. Phage (10 nM in 100 μL of PBS) and 1 μL of K₁₄-alkyne (525 μM in water) were thoroughly mixed by pipetting ˜25 times. For phage wrapped with PEGylated ligands, phage were mixed with 0.75 μL of K₁₄-Cys (525 μM in water). For mixed wrapping on the phage surface, 0.5 μL of K₁₄-alkyne was pre-mixed with 0.75 μL of K₁₄-Cys, and then mixed with 100 μL of 10 nM phage. The solution was shaken at room temperature for 15 min on an orbital shaker. Next, 2 μL of PEGylated ligand (625 μM in water) was added to the appropriate wells. For the dual ligand combinations, the PEGylated ligands were pre-mixed in the desired ratio (a 2:1 molar ratio for example), and then 2 μL of the mixture was added to the appropriate wells. The solutions were gently mixed by pipetting, and incubated overnight at 4° C.

Day 3: Next, the click reaction was performed, as previously described, but with the following modifications [1,2,5]. To buffer the pH, triethylammonium acetate was added to a final concentration of 50 mM, followed by the addition of 1.5 μL of 1 mM azide-functionalized PEG. The solutions were mixed by pipetting. Next, ascorbic acid was added to a final concentration of 1 mM and the solutions were mixed by gently pipetting. Then, copper sulfate was added to a final concentration of 1.5 mM, followed by pipetting to mix the solutions. Water was added to the other wells to maintain consistent phage concentrations. The plate was incubated at room temperature for 30 min.

The wells of the ELISA plate were then incubated with the phage samples. The blocking buffer was removed and the wells were gently washed two times with PBS. Next, the phage solution was added to the respective wells and incubated for 45 min. The phage solution was removed, and the wells were washed three times with 300 μL/well of wash buffer PT (0.05% Tween-20 in PBS), once with PBS, and then incubated with horseradish peroxidase-conjugated anti-M13 antibody (100 μL/well, 1:5000 dilution in PBS) for 40 min. The wells were washed three times with PT and once with PBS. The plate was then developed by incubating with HRP substrate solution (100 μL/well; 1 mg/mL o-phenylenediamine dihydrochloride and 0.02% w/v H₂O₂) in citric acid buffer (50 mM citric acid, 50 mM Na₂HPO₄, pH 5.0). The HRP activity was measured spectrophotometrically at 450 nm using a microtiter plate reader (Bio-Tek). The absorbance at 630 nm was subtracted from the absorbance at 450 nm to eliminate background.

Phage-Based Sandwich ELISA for Cell Capture.

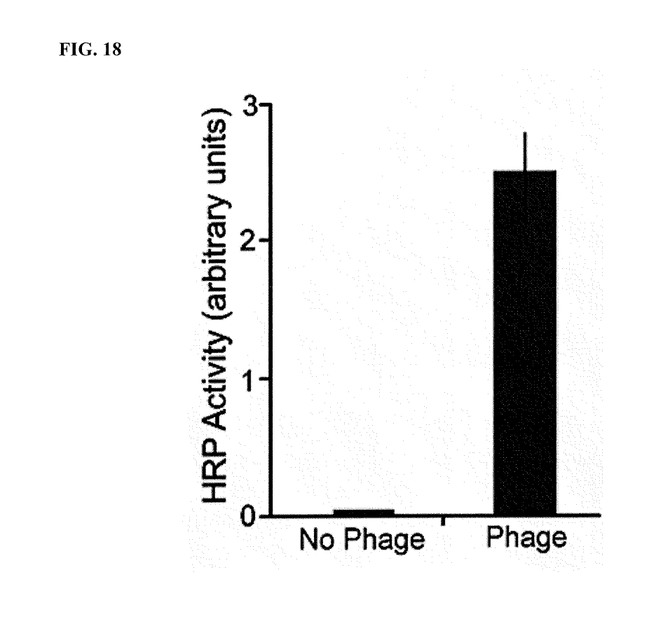

To demonstrate cell capture by the PEGylated-ligand phage, the phage were coated on the plate, and cells added before quantifying binding. This assay setup inverts other cell-based phage ELISAs reported here. This experiment is a significant step towards establishing the relevance of this phage architecture for biosensing assays planned in the future. In this assay, the PEGylated phage architecture is immobilized on the plate as demonstrated in FIG. 18. Next, a cell suspension is added to the wells, and the amount of cells captured are then measured spectrophotometrically as detailed here and in the text. The protocol here focuses on experimental details altered from the above-described ELISA; all other conditions remained unchanged.

Day 1: In this phage capture ELISA, specific wells of a 96-well microtiter plate were coated with 100 μL/well of a solution of 10 nM phage pre-wrapped with oligolysine wrappers, as described above. The plate was incubated for 1 h on a shaker at room temperature. The coating solution was removed, and the wells were blocked with 200 μL/well of 0.2% w/v solution of BSA in PBS for 30 min, and washed two times with PT. Next, 98 μL PBS was added per well, followed by PEGylated ligands and incubated overnight at 4° C.

Day 2: Azide-functionalized PEG variants were then conjugated as described above. Separately, the cells were collected and the concentration adjusted as described above; the ELISA plate was then incubated with 100 μL/well of the cell solution or media for 1 h. The wells were washed with PBS and incubated with 100 μL/well of the anti-PSMA antibody, YPSMA antibody (Abcam) at 1:1000 dilution. The wells were then washed with PBS, followed by incubation with horseradish-peroxidase-conjugated anti-mouse antibody (Sigma) at a 1:1000 dilution. The levels of phage binding were quantified as described above.

### Example 2

### Engineering Chemically Modified Viruses for Prostate Cancer Cell Recognition

Abstract.

Specific detection of circulating tumor cells and characterization of their aggressiveness could improve cancer diagnostics and treatment. Metastasis results from such tumor cells, and causes the majority of cancer deaths. Chemically modified viruses could provide an inexpensive and efficient approach to detect tumor cells and quantitate their cell surface biomarkers. However, non-specific adhesion between the cell surface receptors and the virus surface presents a challenge. This report describes wrapping the virus surface with different PEG architectures, including as fusions to oligolysine, linkers, spacers and scaffolded ligands. The reported PEG wrappers can reduce by >75% the non-specific adhesion of phage to cell surfaces. Dynamic light scattering verified the non-covalent attachment by the reported wrappers as increased sizes of the virus particles. Further modifications resulted in specific detection of prostate cancer cells expressing PSMA, a key prostate cancer biomarker. The approach allowed quantification of PSMA levels on the cell surface, and could distinguish more aggressive forms of the disease.

Introduction.

The migration and dissemination of tumor cells, termed metastasis, causes ≈90% of cancer deaths.[1,2] Metastasis requires loss of apoptotic regulation, and such cells respond poorly to conventional anti-cancer treatments. With a majority of the estimated 27,540 deaths from prostate cancer (PCa) in the US for 2015[3] resulting from metastasis[2], new methods for efficient detection and characterization of metastatic cells could impact clinical care and patient prognosis. Previously, we reported the sensitive detection of soluble prostate-specific membrane antigen (PSMA), a PCa biomarker, at 100 pM concentrations using viruses incorporated into an electrically conductive polymer[4]. Here, we engineer similar bacteriophage, termed ‘phage,’ with polymers and ligands for direct binding to PSMA found on the surface of PCa cells.

PSMA, a 750 residue, 90 kD glycoprotein, is overexpressed on the surface of tumor cells as a non-covalent homodimer in both primary and metastatic cancers [5,6]. Differential splicing during tumorigenesis leads to expression of PSMA as a type II integral membrane protein [7]. Elevated PSMA levels have also been observed in seminal fluid and urine of PCa patients [8]. To detect the protein in urine, we reported viruses with both genetically displayed and chemically synthesized ligands for the sensitive detection of PSMA [4,9]. These ligands, selected from phage-displayed peptide libraries had the following amino acid sequences: ligand-1 (CALCEFLG) (SEQ ID NO:5) and ligand-2 (SECVEVFQNSCDW) (SEQ ID NO:6). Genetically encoded, phage-displayed ligand-2 binds with >100-fold higher affinity to PSMA than ligand-1 [4,10].

Used ubiquitously for molecular display applications, the M13 filamentous phage applied here infects E. coli, and can be manipulated to present genetically encoded peptides on the phage surface [11-13]. The M13 virus consists of a circular, single-stranded DNA genome surrounded by a protein coat composed of approximately 2700 copies of the major coat protein, P8, an α-helical protein of 50 amino acid residues with an unstructured N-terminus. One Glu and two Asp residues near the N-terminus of P8 impart a high negative charge to the outer surface of the virus at physiological pH [14]. Selections with phage-displayed libraries of peptides and proteins can target tissue-cultured cells and even organs in living organisms [15-17]. Phage have also been incorporated into nanomedicine platforms for targeted drug delivery [18-20] and imaging [21,22]. Such applications require low background binding by phage to cell surfaces.

Phage typically adhere to cell surfaces with high affinity, however. Such non-specific adhesion complicates the design of phage-based sensors for the detection of tumor cells; the non-specific background can reduce the signal to noise ratios and the ability to distinguish tumor from non-tumor cells. Francis and co-authors have reported covalently linking the coat proteins of fd phage with both polyethylene glycol (PEG) and imaging agents through a two-step reaction [23]. M13 and fd phage are closely homologous with similar sizes, structural features and sequences [24]. An alternative approach described here, applies non-covalent attachment to the phage surface to access additional architectures for biosensor applications.

Non-covalent attachment offers comparable stability to covalent modification of the virus surface. The high negative charge on the phage surface allows non-covalent wrapping with cationic peptides and polymers [25,26]. Linking these wrappers to recognition ligands opens new routes to greater sensitivity and specificity for target analytes. The peptide ligands can be chemically synthesized and fused to an oligolysine peptide (K₁₄), which ‘wraps’ around the virus particle through complementary electrostatic interactions. Previously, this strategy allowed maximization of ligand density on the phage surface for sensitive detection of biomarkers in complex biofluids, such as synthetic urine v[4]. Here, the overall design incorporates PEG polymers in conjunction with this wrapping strategy to address the problem of non-specific adhesion between phage and cells. Then, we optimize various architectures for the specific detection of PCa cells.

Results and Discussion.

Non-Specific Adhesion of Viruses to Cells.

Among prostate cancer cell lines, LNCaP cells provide the most commonly used in vitro model for early stage PCa [27,28]. Derived from the lymph node adenocarcinoma of the human prostate, LNCaP expresses most of the important PCa biomarkers including PSMA, PSA and AR [29]. Attempts to recognize cell surfaces with conventional phage-displayed ligands resulted in unacceptably high, non-specific adhesion by control phage, which lack a displayed peptide. As shown by ELISA, phage-displayed PSMA ligand 2 and control phage produced similar high levels of binding to LNCaP cells (FIG. 9). In this and essentially all ELISAs reported here, cells are immobilized on microtiter plates; phage are then added before washing away non-binding viruses, and levels of bound phage are quantified spectrophotometrically using an anti-M13 antibody conjugated to horse radish peroxidase (HRP), which catalyzes conversion of its substrate into a colored product. Thus, the high levels of adhesion by both ligand-displayed and control phage are due to non-specific adhesion between phage coat proteins and abundant cell surface receptors, glycans and other molecules. To overcome this non-specific adhesion, we focused on eliminating such interactions by control phage.

Wrapping Phage with PEG to Prevent Non-Specific Adhesion.

The water soluble polymer PEG is commonly bioconjugated to proteins to reduce non-specific adhesion to cells and other surfaces [30-33]. In addition, PEG can increase the solubility of attached therapeutic proteins, prolong circulation times, and decrease proteolysis [34]. Furthermore, the activities of proteins conjugated to PEG typically remain unaffected [35,36]. PEG has been shown to broadly adopt two distinct conformations—descriptively termed ‘mushroom’ and ‘brush.’ [30,37,38] The transition from the mushroom conformation, a more random orientation, to the brush conformation is dependent upon the polymer length and packing densities; longer PEG lengths and higher packing densities favor formation of the brush conformation. This transition can result in a significant drop in non-specific adsorption. In many systems, a mole fraction of 0.15 PEG-modified to -unmodified sites yields significantly reduced non-specific adhesion. High packing densities with such mole fractions can force the polymer to adopt a more stretched, and extended brush conformation to more effectively suppress non-specific adhesion [30]. To provide a framework for experimental design and data interpretation, the reported PEG polymers are assumed to form mushroom and brush conformations based on PEG lengths and packing densities, as has been reported previously [30,37,38].

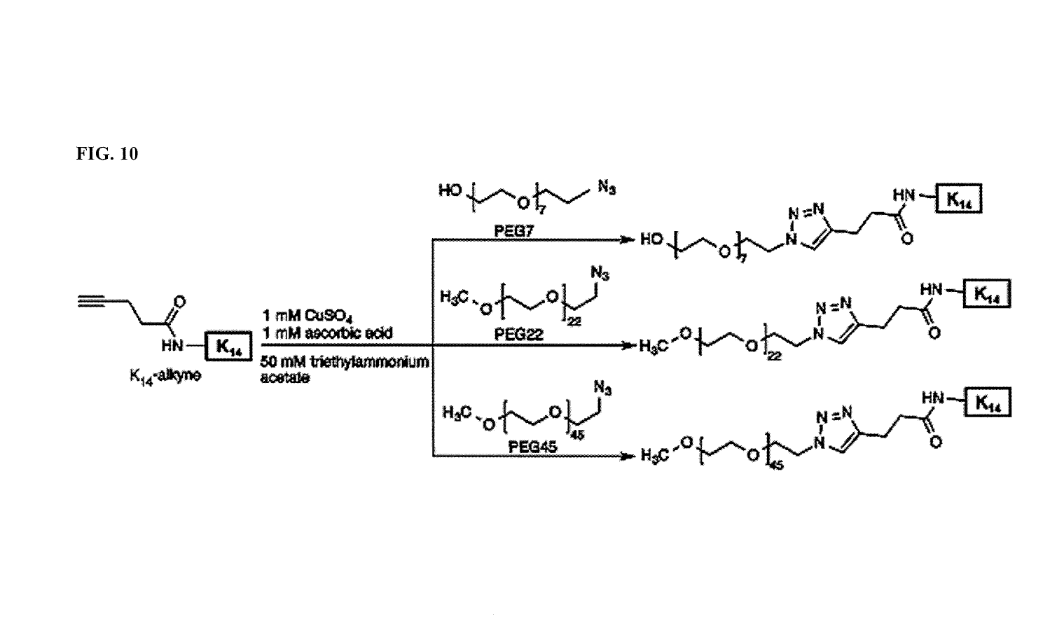

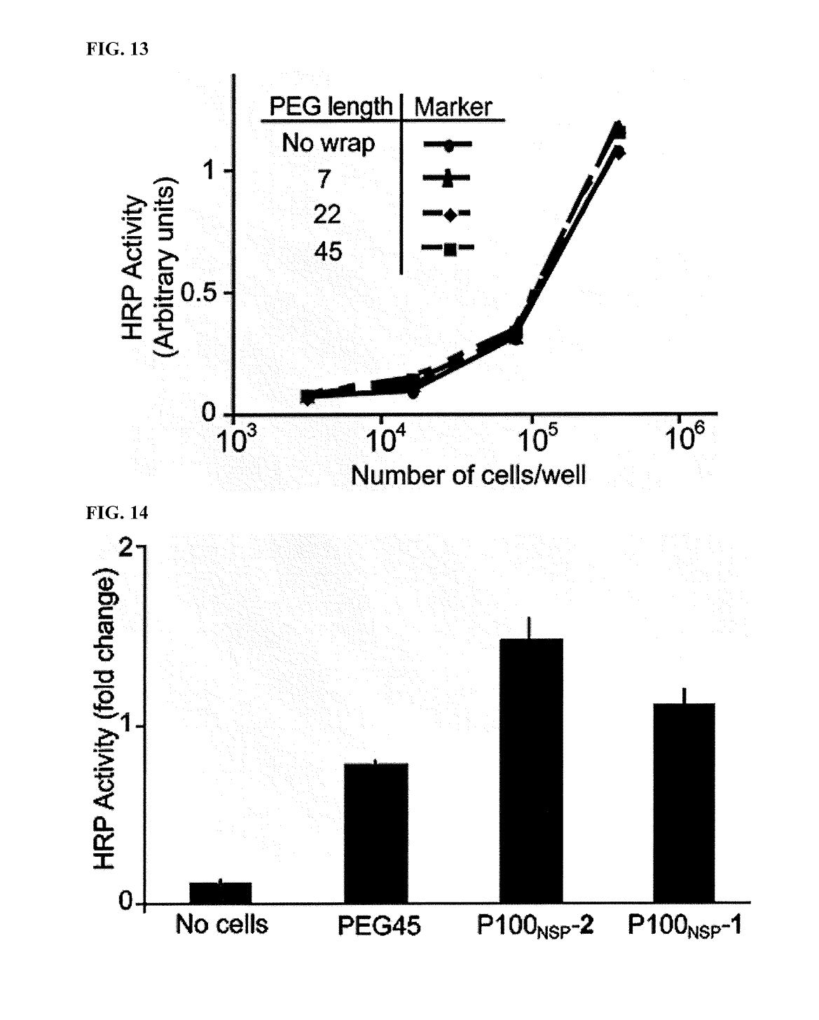

Initial attempts to block non-specific cell adhesion applied PEG variants with different MWs as phage wrappers. Azide-functionalized, polydispersed PEGs with size distributions centered around 7, 22 or 45 ethylene glycol units (providing average MWs of 300, 1K or 2K, respectively) were conjugated to K₁₄-alkyne using the Cu^{I}-catalyzed cycloaddition (‘click’) reaction, FIGS. 10-12. The conjugated peptides were then purified by reverse-phase HPLC and characterized by MALDI-TOF mass spectrometry. The relative adhesion levels of unwrapped and PEG-wrapped phage targeting immobilized LNCaP cells were compared by phage-based ELISA, FIG. 13. Since the phage lacked a displayed peptide, adhesion could only result from non-specific interactions by the phage coat proteins.

In theory, phage wrapped with PEG should bind to LNCaP cells with much lower affinity due to decreased non-specific adhesion. However, no such reduction was observed for the different MW PEGs used, FIG. 13. The ineffectiveness of this initial approach likely resulted from interaction between PEG and the K₁₄ sidechains used to wrap the phage. A crown ether-like encapsulation can form between the primary amine of the Lys sidechains and ethylene glycols of PEG [39], thereby could render K₁₄ incapable of wrapping the phage surface. Without the PEG wrapping the phage surface, the results merely compare phage in different assay wells, as is evident from the overlapping responses. The lack of wrapping by PEGylated oligolysine was further verified by dynamic light scattering measurements, which revealed no significant change in the cross-sectional diameter of the treated phage (data not shown).

On-Phage Cycloaddition Reaction to Generate PEGylated Phage.

To overcome K₁₄ encapsulation by PEG, phage with PEG wrappers were generated in two steps, FIG. 1. First, phage were wrapped with K₁₄-alkyne by incubation at room temperature for 15 min. During this step, the K₁₄-alkyne wrap the phage prior to PEG conjugation. Next, PEG azides were added, and the cycloaddition reaction with the K₁₄-alkyne took place on the phage surface for 30 min. Next, the ELISA described for FIG. 13 was repeated with these PEGylated phage comparing the non-specific adhesion of phage wrapped with different MW PEGs, FIG. 2; in this experiment negative controls of ‘No wrap’ and ‘No cells’ indicate the extremes of high levels of non-specific adhesion to LNCaP cells and non-binding, respectively. Phage wrapped with PEG45 and PEG100 (average MW 2K and 5K, respectively) demonstrate a >75% reduction in non-specific binding to LNCaP cells, as demonstrated by the observed decrease in HRP activity resulting from lower phage binding. The experiment confirms that PEG wrappers can effectively suppress non-specific adhesion, provided the K₁₄ wraps around the phage first. The reduction in non-specificity increases with larger MW PEG polymers, and saturates at around 45 ethylene glycol units.

PEG reduces non-specific binding largely by surrounding the attached surface with a hydration sphere [40]. Direct contact to the phage surface, termed primary adsorption, requires smaller non-specific binding partners to penetrate the PEG layer. Alternatively, the non-specific binding partners could adhere to the outer surface of the PEG layer, termed secondary adsorption. For non-specific adhesion to the larger surfaces of cells, such secondary adsorption is likely a more pronounced effect. To minimize secondary adsorption, the wrappers were applied at 0.15 mole fraction [30]. Here, we estimate the mole fraction as the stoichiometry of PEG molecules added to P8 coat proteins; this analysis is analogous to the calculations for PEG grafted in lipid membranes [41]. Additionally, we assumed that at the concentration used, PEG22, 45 and 100 adopt brush conformations due to their high packing densities [30,37], which were fixed by maximization of oligolysine wrappers as previously described [4].

Based on published precedent, PEG7 presumably adopts a mushroom conformation [30], and fails to suppress non-specific adhesion to the same levels. The brush conformation of the larger PEGs can more efficiently reduce non-specific secondary adsorption due to the hydration sphere extending further from the virus surface. Beyond a certain height of the polymer brush, the effect of secondary adsorption remains constant as shown by the nominal difference obtained between PEG45 and 100 in FIG. 2. For ligand-based recognition described further below, phage wrapped with PEG45 provided the negative control phage.

Dynamic Light Scattering (DLS) Analysis of PEGylation.

To characterize the PEG-wrapped phage, DLS measurements were conducted, FIGS. 3A-3B. The M13 phage used here have dimensions of approximately 6 by 1000 nm [42]. Rayleigh scattering provides an estimated 45.9 nm diameter of the average cross-section; this experiment uses measurement with backscatter mode, having a scattering angle of 173°, for unwrapped and unmodified phage. For comparison, the comparable reported measurement with covalently and genetically modified fd phage yielded a reported average cross-sectional diameter of 70 nm [43]. Due to the filamentous nature of the phage as a long, flexible cylinder, such values can only provide a relative change in size. Furthermore, the forward scatter mode (scattering angle of 13°) provides a 715 nm average size for the M13 phage applied here, which compares well with previously reported 650 nm average size for fd phage [43]. Since the phage length remains roughly unchanged with wrapping, we found negligible difference in the average phage sizes measured by forward scattering, and instead focused on DLS measurement in backscatter mode.

Next, the change in average cross-sectional diameter was measured for different samples from each step of the phage wrapping process, FIG. 3A. The addition of K₁₄-Cys wrappers on the phage leads to an increase in cross-sectional diameter from 45.9 to 50.0 nm. Upon conjugation of this K₁₄-Cys wrapped phage to maleimide-functionalized. PEG100, an approximate 10 nm increase in size is observed. This increase in size matches two independent reports for size increases after PEG100 bioconjugation to gold nanoparticles [44,45]. Thus, the DLS-based size measurements confirm the formation of the expected phage-wrapped complexes.

Synthesis of PEGylated Ligands.

Towards the goal of specific recognition of a cell surface receptor, different scaffolds for the display of ligands on phage were explored. First, heterobifunctional PEG, Mal-PEG-NH₂, provided reactive groups for selective attachment of oligolysine at the maleimide end and PSMA binding ligands to the amine end, Scheme 1. As the scaffold, PEG100 was chosen to provide a longer polymer brush to reduce non-specific secondary adsorption. Azide-functionalized PSMA ligands 1 and 2 were synthesized by conventional solid-phase peptide synthesis (SPPS), and coupled to pentynoic acid via the click reaction. The resultant N-terminal carboxylic acid group was then coupled to Mal-PEG100-NH₂ using HBTU as an amide bond forming agent in water. Since this reaction non-specifically couples amine and carboxylate functionalities, the attachment sites could vary as both ligands have sidechain carboxylic acids. The resultant ligand is termed P100_{NSP}-1/2 for ‘PEG100, non-specific attachment to ligand 1 or 2.’ as described in Table 1.

Subsequently, phage wrapped with K₁₄-Cys were coupled to the maleimide terminus of P100_{NSP}-1/2, as described above. Preliminary validation of binding to cell surface PSMA by the PEGylated ligand wrappers was performed by phage ELISA as before, FIG. 14. Compared to the non-specific binding observed in FIG. 9, a slight improvement in binding affinity resulted from wrapping with the PEGylated ligands. This modest result provided a starting point for further engineering. Phage wrapped with P100_{NSP}-2 displayed a higher affinity for LNCaP cells compared to P100_{NSP}-1 as expected from its higher binding affinity for PSMA.

Bidentate Binding Mode of PEGylated Ligands.

Dual display of ligands 1 and 2 can enable synergistic, high affinity binding to PSMA due to a bidentate binding mode and a hook and loop-like avidity effect [4]. A phage ELISA targeting LNCaP cells with different ratios of the two PEGylated, phage-wrapped ligands examined relative binding affinities. First, the effectiveness of the bidentate binding mode (FIG. 4) was compared to binding by individual ligands (patterned bars in FIG. 4). Having two ligands on the phage surface consistently improved binding affinity. Furthermore, a 2:1 mixture of P100_{NSP}-2 and P100_{NSP}-1 respectively, was found to maximize the recognition of PSMA on LNCaP cells. The improved binding from a 2:1 ratio stems from the higher binding affinity of ligand 2 compared to ligand 1. Altering this ratio in either direction drops the apparent affinity, likely due to loss of optimal bidentate binding. Hereafter, phage were wrapped with a 2:1 mixture of the PEGylated ligands 2 and 1, respectively.

Optimizing the Attachment Site for PEGylated Ligands.

Further optimization explored the size, geometry and attachment site of the ligands fused to the PEG wrapper. Such variables can be crucial to the pharmacokinetic properties of PEGylated drugs, which demonstrates the sensitivity of biological recognition to such factors [46]. For example, the attachment sites of the peptide ligand to PEG100 dictates the ligand orientation and the potential availability of peptide sidechains. An alternative synthesis scheme was designed to control ligand orientation. Mal-PEG100-NH₂ was first coupled to pentynoic acid, FIG. 15. The resultant Mal-PEG100-alkyne was then coupled to the azide-functionalized peptide ligands using click chemistry, providing a specific site of attachment to the ligand. The resulting PEGylated ligand is termed P100_{SP}-1/2 for ‘PEG100, specific attachment to ligand 1 or 2,’ as described in Table 1.

TABLE 1
Nomenclature of PEGylated PSMA ligands. All ligands
were bioconjugated to phage wrapped with K₁₄-Cys.

| PEG | | PEG4 | |
|---|---|---|---|
| length | Attachment | linker | Nomenclature |
| P100 | NSP | — | P100_{NSP}-X |
| P100 | SP | — | P100_{SP}-X |
| P100 | NSP | ✓ | P100_{NSP}-P4-X |
| P100 | SP | ✓ | P100_{SP}-P4-X |

SP: Specific;
NSP: Non-specific
X = ligand 1 (CALCEFLG) or 2 (SECVEVFQNSCDW)

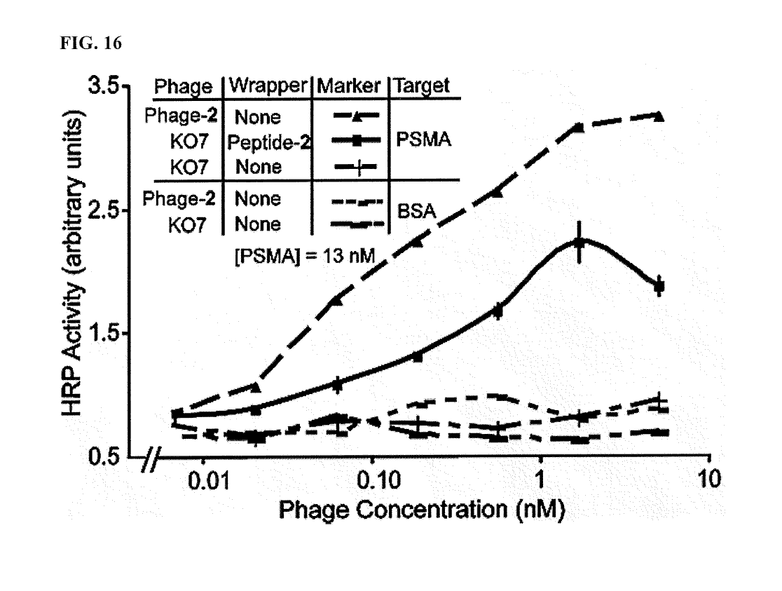

Specific attachment of PEG to the wrapped ligands could improve binding affinity by removing attachment through the ligands' sidechains and also altering their orientation on the phage surface. The significance of ligand orientation is apparent through the higher binding affinity observed for genetically encoded, phage-displayed ligand 2 (dashed red line) relative to phage wrapped with chemically synthesized ligand 2 (solid line), FIG. 16. When genetically displayed on the phage, ligand 2 has a free N-terminus, but the synthesis of P100_{SP}-2 inverts this orientation, leaving a free C-terminus, and an N-terminus directly conjugated to the triazole and then PEG100 (as shown in the schematic flowchart of FIG. 17). As attained by the specific attachment of P100_{SP}-2, the N-terminal Glu residue of ligand 2 requires an unhindered and unmodified carboxylate sidechain, as previously shown by homolog shotgun scanning [10]. The sidechain of Glu2 could be partially modified in P100_{NSP}-2 due to non-specific attachment through the carboxylate sidechain. Subsequent experiments compared bioconjugation to either the N-terminal azide or carboxylate sidechain through incorporation of an additional linker.

Insertion of a PEG4 Linker to Reduce Steric Effects on the Attached Ligands.

Heterobifunctional linkers between PEG and a molecule of interest can enhance activity through flexible additional spacing [40]. We envisioned the incorporation of an average 175 MW PEG4 linker between the peptide ligand and the triazole generated by the click reaction could enhance the binding affinity of the peptide ligands. With only four ethylene glycol units, this highly flexible linker can disconnect the peptide ligand from any steric constraints dictated by PEG100 or the triazole, FIG. 17. Thus, the peptide ligands were resynthesized via SPPS, and coupled to azido-PEG4-carboxylic acid (15-Azido-4,7,10,13-tetraoxapentadecanoic acid), thereby inserting a PEG4 linker before the azide functionality. Azido-PEG4-ligands were further linked to PEG100 following the two synthesis routes described above, specific and non-specific addition. The resultant PEGylated ligands are termed P100_{SP}-P4-1/2 and P100_{NSP}-P4-1/2, P4 to indicate the insertion of the PEG4 linker, Table 1. The P100_{SP}-P4-1 and P100_{SP}-P4-2 conjugated peptides were shown to have the expected sizes by gel permeation chromatography (Supplementary Information) and DLS. A further increase of 10 nm in cross-sectional diameter was observed for the addition of the PEG4-fused ligand (FIG. 3B).

An ELISA compared the relative binding affinities of the four PEGylated ligand 2 variants—specific (solid) and non-specific (patterned) attachment with and without the PEG4 linker, FIG. 5A. P100_{SP}-2 demonstrates a higher binding affinity for cell surface PSMA than P100_{NSP}-2, illustrating the significance of the unmodified Glu sidechain obtained through specific attachment. Furthermore, inclusion of the PEG4 linker further enhances the binding affinity for both P100_{SP}-2 and P100_{NSP}-2. As a result, the PEGylated ligand P100_{SP}-P4-2 incorporating the PEG4 linker with specific attachment site provided the most effective architecture for the PEGylated ligand to recognize PSMA on the cell surface.

The dual ligand combinations of peptides 1 and 2 were expected to further provide higher affinity through bidentate binding. However, only a modest improvement was observed for the combination of P100_{NSP}-2+P100_{NSP}-1 versus the best individual ligand, P100_{SP}-P4-2, FIG. 5B. The slightly greater binding affinity can be attributed to the bidentate binding mode of the dual ligand system. Furthermore, the architecture of the PEG4 (P4) linker also required optimization. The geometry of the PEG4 linker clearly affects the availability of the two Lys sidechains in the 8-mer peptide 1, as shown by the drop in affinity for P100_{NSP}-P4-2+P100_{NSP}-P4-1. This reduction in apparent binding affinity could be due to the formation of a crown ether-like cavity by PEG4, which naturally adopts a mushroom-like conformation based on its size [30]. Furthermore, the combination has affinity equivalent to P100_{NSP}-P4-2, which indicates complete loss of ligand 1 activity by PEG4 masking; this effect renders the dual ligand combination of P100_{NSP}-P4-2+P100_{NSP}-P4-1 equivalent to the individual ligand, P100_{NSP}-P4-2. Notably, ligand 2 lacks Lys residues, and is therefore not susceptible to such masking effects.

Controlling the geometry of the PEG4 linker could prevent masking of the Lys sidechains of ligand 1. Sandwiching PEG4 between PEG100 and the peptide ligand through the specific attachment mode, eliminates such debilitating effects, as shown by a significant increase in binding affinity for the dual ligand system P100_{SP}-P4-2+P100_{SP}-P4-1 (FIGS. 5B and 17). This specific attachment incorporating the PEG4 linker evidently stretches the PEG4 providing higher apparent affinity from a constitutional isomer with different geometry. Thus, in the next experiments, phage were wrapped with the dual ligand combination of P100_{SP}-P4-2+P100_{SP}-P4-1 in a 2:1 ratio.

PEG Spacers to Control Relative Ligand Spacing.

The relative spacing between ligands governs the synergy of the chelate-based avidity effect. To achieve optimal geometry of the two ligands, the relative spacing was systematically engineered by interspersing long PEGylated ligands with smaller PEG wrappers on the phage surface. The smaller PEG wrappers could provide spacers to push apart the PEG-fused ligands on the phage surface. Generating ligands and spacers required the two wrapping modes described above, click chemistry and cysteine-maleimide reaction, on the same phage. K₁₄-alkyne and K₁₄-Cys were pre-mixed to an estimated mole fraction of 0.19 (as described above), and then used to wrap the phage surface. K₁₄-alkyne was linked to short PEG polymers to provide spacers. Different concentrations of the PEG polymers were explored. The ratio of ligands to spacers was empirically optimized, and a ratio of 1.5:1 provided the best levels of PSMA recognition (data not shown). The concentration of the PEGylated ligands remained unchanged, and a 2:1 molar mixture of the two ligands was reacted with the K₁₄-Cys wrapped on the phage surface, A higher net concentration of wrappers could be accommodated by the phage as the spacers allowed higher packing density.

The dual PSMA ligand combination described above, P100_{SP}-P4-2+P100_{SP}-P4-1, without (green) or with spacers (brown) of either PEG 7, 22 or 45, wrapped around the phage were assayed for binding to LNCaP cells, FIG. 6. All spacers significantly enhanced PSMA recognition by the displayed ligands. However, the PEG7 spacer proved most effective. The much smaller PEG7 spacer can force the ligands into adopting a more optimal geometry for effective bidentate binding, and the height of this polymer brush does not interfere with ligand binding. Longer spacers failed to boost binding affinity to the same levels. At the mole fraction of PEG used, the PEG polymers can adopt the brush conformation with the height of the polymer brush dependent on the PEG length. Interdigitation of PEG spacers with PEGylated ligands can interfere with the binding affinity of the ligands, as shown with the longer brushes of PEG22 and 45. Also, the addition of K₁₄-alkyne without conjugated PEG spacers has no effect on binding affinity, as expected; thus, the increased packing of oligolysine wrappers is not a contributing factor. Rather, enhanced binding results from the improved geometry through addition of PEG spacers.

Selective Recognition of PSMA Positive Cells.

To demonstrate specificity for PCa cells by these chemically modified phage, binding to different prostate cancer cell lines was compared. LNCaP cells can model early or late stage cancer cells, through variation in their culture conditions. The majority of PCa cases gain resistance to therapies based on androgen ablation [47]. The LNCaP cell line, a model for early stage PCa, is androgen sensitive but gradually loses the androgen requirement, providing a model for late stage PCa, which also mimics androgen ablation [47,48]. The latter can be simulated by culturing LNCaP cells in androgen-depleted media, referred to as LNCaP CSS (for charcoal-stripped serum) [49,50]. Increased levels of PSMA are associated with androgen independent PCa [48]. Thus, both LNCaP and LNCaP CSS cell lines were assayed. The third cell line, PC3 cells, do not express PSMA, and were used as the negative control [29,51]. The following assays validate the dual ligand system for cell line discrimination and quantification of cell surface receptors.

The optimized dual ligand combination of P100_{SP}-P4-2+P100_{SP}-P4-1 and the PEG7 spacer was assayed for binding to LNCaP, LNCaP CSS, and PC3 cell lines, FIG. 7. The results demonstrate high specificity for PSMA positive LNCaP cells in a dose-dependent manner with higher apparent affinity to LNCaP CSS cells. This higher sensitivity to LNCaP CSS cells is consistent with the increase in PSMA expression resulting from the progression of the cancer cells to an androgen independent state in the LNCaP CSS model [48].

Detecting PSMA on Suspended Cells and in Culture Media.

The tailored phage could also capture cells from solution, which is critical for future analytical applications in circulating tumor cell detection and characterization. In this experiment unlike other ELISAs described here, the phage were immobilized on the microtiter plate before applying a solution of cells, FIGS. 8 and 18; levels of bound cells were quantified through application of anti-PSMA primary antibody and HRP-conjugated, anti-mouse, secondary antibody. Again, phage wrapped with the dual ligand combination of P100_{SP}-P4-2+P100_{SP}-P4-1 and the PEG7 spacer were used. In this experiment, the capture of PSMA positive cells is detected by and proportional to cell surface PSMA concentration. PC3 cells, lacking PSMA, do not generate a significant response, as expected.

PSMA levels are elevated in the urine samples of PCa patients, and levels of this biomarker correlate with the aggressiveness of the disease [8,52]. Therefore, cultured PCa cells should release PSMA into their culture media. Thus, PSMA detection was also performed with cell culture supernatant, normalized to the volume and the number of cells (FIG. 8). The PEGylated dual ligand combination on phage allows sensitive PSMA detection in 100 μL of supernatant from both LNCaP and LNCaP CSS cell cultures. Cell culture media from PC3 cells and fresh culture medias serve as the negative controls. As expected, the negative controls failed to show any significant binding. The effective detection of PSMA shed by LNCaP cells, in androgen sensitive and androgen independent cells, demonstrates the use of phage wrapped with PEGylated ligands for future development of analytical devices and translation to the clinic.

### REFERENCES

[1] S. A. Joosse, T. M. Gorges and K. Pantel, EMBO Mol. Med., 2015, 7, 1-11; [2] P. Mehlen and A. Puisieux, Nat. Rev. Cancer, 2006, 6, 449-458; [3] R. L. Siegel, K. D. Miller and A. Jemal, C A. Cancer J. Clin., 2015, 65, 5-29; [4] K. Mohan, K. C. Donavan, J. A. Arter, R. M. Penner and G. A. Weiss, J. Am. Chem. Soc., 2013, 135, 7761-7767; [5] N. Schülke, O. A. Varlamova, G. P. Donovan, D. Ma, J. P. Gardner, D. M. Morrissey, R. R. Arrigale, C. Zhan, A. J. Chodera, K. G. Surowitz, P. J. Maddon, W. D. W. Heston and W. C. Olson, Proc. Natl. Acad. Sci. USA, 2003, 100, 12590-12595; [6] A.-Y. Chuang, A. M. DeMarzo, R. W. Veltri, R. B. Sharma, C. J. Bieberich and J. I. Epstein, Am. J. Surg. Pathol., 2007, 31, 1246-1255; [7] M. Kawakami and J. Nakayama, Cancer Res., 1997, 57, 2321-2324; [8] R. L. Sokoloff, K. C. Norton, C. L. Gasior, K. M. Marker and L. S. Grauer, Prostate, 2000, 43, 150-157; [9] K. Mohan and G. A. Weiss, Anal. Biochem., 2014, 453, 1-3; [10] J. A. Arter, J. E. Diaz, K. C. Donavan, T. Yuan, R. M. Penner and G. A. Weiss, Anal. Chem., 2012, 84, 2776-2783; [11] G. P. Smith, Science, 1985, 228, 1315-1317; [12] S. S. Sidhu and G. A. Weiss, Phage Display: A Practical Approach, Oxford University Press, New York, 2004; [13] J. K. Scott and G. P. Smith, Science, 1990, 249, 386-90; [14] L. C. Welsh, M. F. Symmons, J. M. Sturtevant, D. A. Marvin and R. N. Perham, J. Mol. Biol., 1998, 283, 155-177; [15] M. Trepel, W. Arap and R. Pasqualini, Curr. Opin. Chem. Biol., 2002, 6, 399-404; [16] G. Abbineni, S. Modali, B. Safiejko-Mroczka, V. A. Petrenko and C. Mao, Mol. Pharm., 2010, 7, 1629-1642; [17] W. Arap, R. Pasqualini and E. Ruoslahti, Science, 1998, 279, 377-380; [18] V. A. Petrenko and P. K. Jayanna, FEBS Lett., 2014, 588, 341-349; [19] P. K. Jayanna, V. P. Torchilin and V. A. Petrenko, Nanomedicine., 2009, 5, 83-89; [20] T. Wang, S. Yang, V. A. Petrenko and V. P. Torchilin, Mol. Pharm., 2010, 7, 1149-1158; [21] D. Ghosh, Y. Lee, S. Thomas, A. G. Kohli, D. S. Yun, A. M. Belcher and K. A. Kelly, Nat. Nanotechnol., 2012, 7, 677-682; [22] D. Ghosh, A. F. Bagley, Y. J. Na, M. J. Birrer, S. N. Bhatia and A. M. Belcher, Proc. Natl. Acad. Sci. U.S.A, 2014, 111, 13948-13953; [23] Z. M. Carrico, M. E. Farkas, Y. Zhou, S. C. Hsiao, J. D. Marks, H. Chokhawala, D. S. Clark and M. B. Francis, ACS Nano, 2012, 6, 6675-6680; [24] M. Russel, H. B. Lowman and C. Tim, Phage Display: Practical Approach, Oxford University Press, New York, 2004; [25] J. A. Lamboy, P. Y. Tam, L. S. Lee, P. J. Jackson, S. K. Avrantinis, H. J. Lee, R. M. Corn and G. A. Weiss, Chem Bio Chem, 2008, 9, 2846-2852; [26] J. A. Lamboy, J. A. Arter, K. A. Knopp, D. Der, C. M. Overstreet, E. F. Palermo, H. Urakami, T.-B. Yu, O. Tezgel, G. N. Tew, Z. Guan, K. Kuroda and G. A. Weiss, J. Am. Chem. Soc., 2009, 131, 16454-16460; [27] M. G. Dozmorov, R. E. Hurst, D. J. Culkin, B. P. Kropp, M. B. Frank, J. Osban, T. M. Penning and H.-K. Lin, Prostate, 2009, 69, 1077-1079; [28] J. S. Horoszewicz, S. S. Leong, T. M. Chu, Z. L. Wajsman, M. Friedman, L. Papsidero, U. Kim, L. S. Chai, S. Kakati, S. K. Arya and A. A. Sandberg, Prog. Clin. Biol. Res., 1980, 37, 115-132; [29] R. E. Sobel and M. D. Sadar, J. Urol., 2005, 173, 342-359; [30] D. Marsh, R. Bartucci and L. Sportelli, Biochim. Biophys. Acta—Biomembr., 2003, 1615, 33-59; [31] J. Crawford, Cancer Treat. Rev., 2002, 28, 7-11; [32] K. Knop, R. Hoogenboom, D. Fischer and U. S. Schubert, Angew. Chem. Int. Ed. Engl., 2010, 49, 6288-6308; [33] S. S. Pai, T. M. Przybycien and R. D. Tilton, Langmuir, 2010, 26, 18231-18238; [34] C. S. Fishburn, J. Pharm. Sci., 2008, 97, 4167-4183; [35] C. Loo, A. Lin, L. Hirsch, M.-H. Lee, J. Barton, N. Halas, J. West and R. Drezek, Technol. Cancer Res. Treat., 2004, 3, 33-40; [36] C. Loo, A. Lowery, N. Halas, J. West and R. Drezek, Nano Lett., 2005, 5, 709-711; [37] C. S. Levin, S. W. Bishnoi, N. K. Grady and N. J. Halas, Anal. Chem., 2006, 78, 3277-3281; [38] X. Xia, M. Yang, Y. Wang, Y. Zheng, Q. Li, J. Chen and Y. Xia, ACS Nano, 2012, 6, 512-522; [39] M. Sarma, T. Chatterjee and S. K. Das, RSC Adv., 2012, 2, 3920-3926; [40] R. B. Greenwald, Y. H. Choe, J. McGuire and C. D. Conover, Adv. Drug Deliv. Rev., 2003, 55, 217-250; [41] G. Montesano, R. Bartucci, S. Belsito, D. Marsh and L. Sportelli, Biophys. J., 2001, 80, 1372-13783; [42] M. J. Glucksman, S. Bhattacharjee and L. Makowski, J. Mol. Biol., 1992, 226, 455-470; [43] J. Kim, N. Korkmaz and C. H. Nam, IBC, 2012, 4, 1-7; [44] Y. Liu, M. K. Shipton, J. Ryan, E. D. Kaufman, S. Franzen and D. L. Feldheim, Anal. Chem., 2007, 79, 2221-2229; [45] J. Manson, D. Kumar, B. J. Meenan and D. Dixon, Gold Bull., 2011, 44, 99-105; [46] J. M. Harris, N. E. Martin and M. Modi, Clin. Pharmacokinet., 2001, 40, 539-551; [47] C. Tovar, B. Higgins, K. Kolinsky, M. Xia, K. Packman, D. C. Heimbrook and L. T. Vassilev, Mol. Cancer, 2011, 10, 49-59; [48] S. R. Denmeade, L. J. Sokoll, S. Dalrymple, D. M. Rosen, A. M. Gady, D. Bruzek, R. M. Ricklis and J. T. Isaacs, Prostate, 2003, 54, 249-257; [49] Z. Culig, J. Hoffmann, M. Erdel, I. E. Eder, A. Hobisch, A. Hittmair, G. Bartsch, G. Utermann, M. R. Schneider, K. Parczyk and H. Klocker, Br. J. Cancer, 1999, 81, 242-251; [50] Y. Iwasa, A. Mizokami, S. Miwa, K. Koshida and M. Namiki, Int. J. Urol., 2007, 14, 233-239; [51] A. Ghosh, X. Wang, E. Klein and W. D. W. Heston, Cancer Res., 2005, 65, 727-731; [52] S. L. Su, I. P. Huang, W. R. Fair, C. T. Powell and W. D. Heston, Cancer Res., 1995, 55, 1441-1443.

Materials and Methods.

All chemicals and reagents were purchased from Sigma-Aldrich, and used as received unless otherwise noted. PSMA and the cell lines LNCaP and PC3 were generous gifts from Drs. William Ernst and Gary Fuji (Molecular Express). Maleimide-PEG100-amine and 15-azido-4,7,10,13-tetraoxapentadecanoic acid (azido-PEG4-carboxylic acid) were purchased from Alfa Aesar. N,N-Diisopropylethylamine (DIPEA) and azide-functionalized PEG7, 22 and 45 were purchased from Sigma, and 4-Azidobutanoic acid was purchased from Synthonix. 4-Pentynoic acid (GFS Chemicals, Inc.), O-benzotiazole-N,N,N′,N′-tetramethyl-uronium-hexafluoro-phosphate, HBTU (GL Biochem Ltd.), triethyammonium acetate buffer (Fluka Biochemika) and Tween-20 (EMD Science) were used as received. HPLC-grade water was used for the preparation of solutions.

M13 Bacteriophage Propagation.

Phage propagation and isolation was performed as previously described [1,2]. Briefly, the phagemid DNA was transformed into CaCl₂ competent E. coli XL-1 Blue cells. The cells were grown at 37° C. in 2 mL 2YT media supplemented with carbenicillin and tetracycline until the culture reached log-phase growth. The culture was then infected with KO7 helper phage with a multiplicity of infection of 4.5:1. The starting culture was then transferred to 75 mL 2YT media supplemented with carbenicillin and kanamycin. The phage culture was incubated for 16 h at 37° C. with shaking. The phage were isolated from the culture supernatant by centrifugation at 10 krpm after precipitation through addition of ⅕^{th} volume of PEG-NaCl (2.5 M NaCl, 20% PEG-8000). After second precipitation, the phage were resuspended in phosphate-buffered saline (PBS, 135 mM NaCl, 2.50 mM KCl, 8.00 mM Na₂HPO₄, 30.0 mM KH₂PO₄, pH 7.2). Phage concentration was determined by UV absorbance at 268 nm (OD₂₆₈ of 1.0=8.31 nM phage).

Solid Phase Peptide Synthesis.

The peptides were synthesized by conventional solid-phase peptide synthesis with Fmoc-protected amino acids on Rink-amide resin (Novabiochem), as previously described [1,3,4]. The peptide N-terminus was coupled to 4-azido butanoic acid or 4-pentynoic acid to yield the azide- or alkyne-functionalized peptides, respectively. For incorporation of the PEG4 linker, the last coupling step was performed with 15-azido-4,7,10,13-tetraoxapentadecanoic acid. The reported peptides were purified by reverse-phase HPLC purification with a C₁₈ column. Fractions containing the purified peptides were combined and concentrated using rotary evaporation, followed by lyophilization and characterization by MALDI-TOF mass spectrometry. The calculated m/z for peptide-1 [M⁺] 1349.67, found 1349.77. The calculated m/z for peptide-2 [M⁺] 2040.28, found 2040.23. The calculated m/z for peptide-1 fused to the azido-PEG4 linker is [M⁺] 1510.80, found 1511.78. The calculated m/z for peptide-2 fused to the azido-PEG4 linker is [M⁺] 2201.02, found 2202.06. The calculated m/z for alkyne-functionalized K₁₄ peptide [M+Na]⁺1914.37, found 1914.18. The calculated m/z for K₁₄-Cys peptide [M³⁺] 638.79, found 638.79.

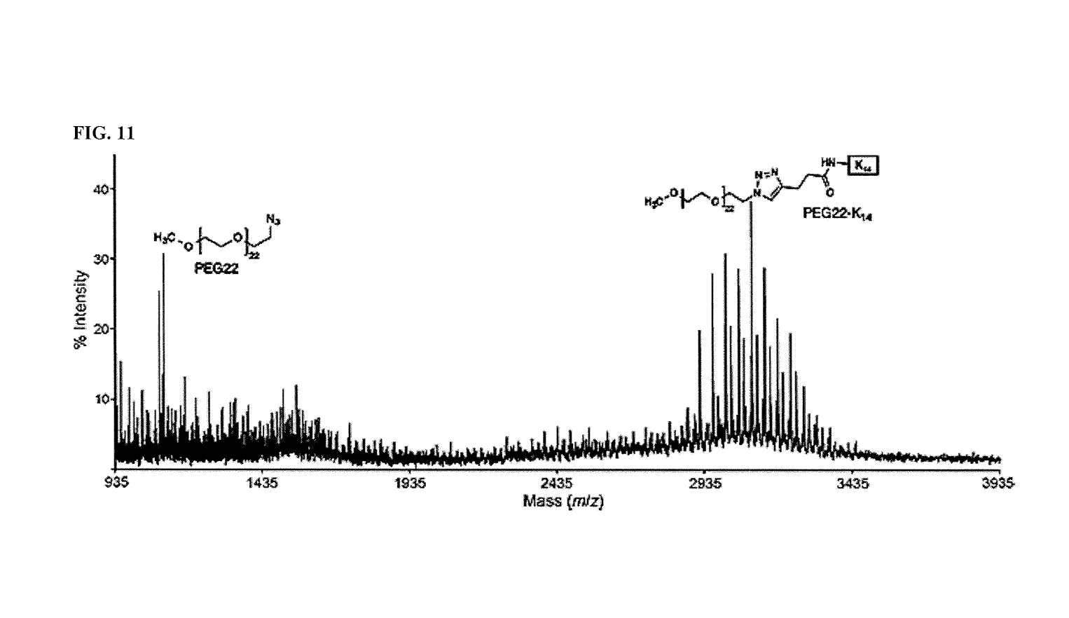

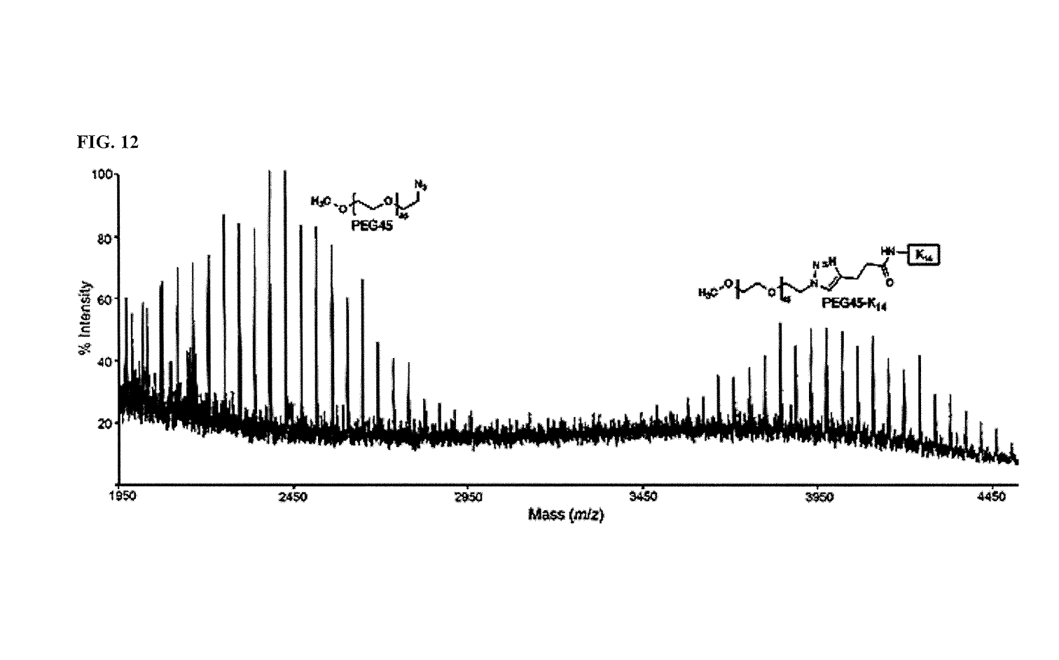

Click chemistry reaction for the synthesis of PEGylated oligolysines. The protocol for the synthesis of PEGylated oligolysines was adapted from Lumiprobe Corporation's protocol, as described previously [1,2,5,6]. Briefly, the reaction was performed at a final concentration of 100 μM azide-functionalized PEG. The product obtained was purified using reverse-phase analytical HPLC and characterized by MALDI-TOF mass spectrometry. The calculated m/z for alkyne-functionalized K₁₄ fused to azide-functionalized PEG7 [M⁺] 2285.61, found 2286.93. The mass spectrometry data obtained for PEGylated oligolysine showed a shift in the characteristically polydispersed PEG spectra by the expected mass of K₁₄-alkyne, FIGS. 11-12.

Synthesis of PEGylated Ligands—Specific Attachment Mode.

The protocol for the synthesis of the PEGylated ligands was adapted from the solid phase peptide synthesis and click chemistry reaction described above, and also described in FIGS. 15 and 17. In a glass test tube, 40 μL of 1 mM Mal-PEG100-NH₂ (commercially purchased from Alfa Aesar) in water, 12 μL of 10 mM pentynoic acid (in water), 12 μL of 10 mM HBTU (in NMP), 40 μL of DIPEA and 296 μL of HPLC grade water were combined and stirred at room temperature for 2 h, yielding alkyne-functionalized-PEG100-Mal. To obtain more quantities of the product, multiple reactions at this volume were run in parallel. To remove unreacted starting material and to concentrate the product, the reaction mixtures were diluted with an equal volume HPLC grade water before concentration to ⅕th volume using 2K MWCO concentrators (Sartorius).

For the next step of the synthesis, ˜50 μL of the alkyne-functionalized-PEG100-Mal, obtained as described above, was conjugated to azide-functionalized peptide ligands (final concentration of 40 μM) by click chemistry as before [1,5]. The reaction mixture was stirred overnight at room temperature. Four identical reactions were run in parallel. To remove unreacted starting materials, the four reaction mixtures were then combined and concentrated to ¼^{th} volume using 3K MWCO concentrators. Next, the resultant solution was diluted with an equal volume of HPLC grade water before concentrating to ˜½ volume using 5K MWCO concentrators. The concentrated reaction mixture was purified using reverse-phase analytical HPLC (FIGS. 19-20) and fractions were identified by MALDI-TOF mass spectrometry. The high polydispersity of the high MW PEG polymer prevents accurate mass determination by this technique, and instead Gel Permeation Chromatography (GPC) was used as described in the next section.

For the non-specific attachment mode described in the text and FIG. 17, the PEGylated ligands were synthesized in the reverse order. The azide-functionalized peptides were first conjugated to pentynoic acid using click chemistry. The resultant peptide was then coupled to Mal-PEG100-NH₂ using HBTU and DIPEA as described above.

Gel Permeation Chromatography (GPC).

GPC was used to characterize the MW's of the PEGylated peptides. The molecular weights of the polymers, calibrated with PEG MW standards, were obtained with an Agilent 1100 series GPC system (Agilent Technologies, Santa Clara, Calif.) using 0.1% (v/v) LiBr/DMF solution (1.0 mL/min) as the eluent. The commercially purchased, unmodified Mal-PEG100-NH₂ was found to have an average molecular weight of Mn=3690, and this polymer eluted as a broad peak consistent with its size distribution. The calculated molecular weights for PEGylated-1 and -2 with the PEG4 linker, based on the molecular weight of Mal-PEG100-NH₂, were estimated as 5300 and 5990, respectively. The corresponding molecular weights observed by GPC were 5310 and 6060, respectively.

Dynamic Light Scattering (DLS).

To demonstrate wrapping by PEGylated ligands and other materials on the phage surface, DLS measurements were obtained using Nano ZetaSizer ZS series. For determination of size, 1 mL of each sample was measured at the same concentration as used for the biological assay. Each sample was measured at least three times at 25° C., with each individual size measurement being the average of 10 runs.

Cell Growth.

The cell lines were grown as monolayers in media supplemented with 10% fetal bovine serum (Cellgro), 1 mM sodium pyruvate [7] and 1% penicillin-streptomycin-glutamine in a 5% CO₂ and 95% air-humidified atmosphere at 37° C. LNCaP cells were cultured in RPMI 1640 media. For studies with LNCaP cells cultured in charcoal-stripped serum (LNCaP CSS cells), LNCaP cells were washed with PBS and then incubated with phenol free RPMI 1640 media supplemented with 10% charcoal-stripped serum (Cellgro) for five minutes. The cells were again washed with PBS, and provided fresh media [8]. PC3 cells were grown in Ham's F-12 media.

Cell-Based Phage Enzyme-Linked Immunosorbent Assay (ELISA):

Day 1: The cell-based ELISA was performed as previously described by Watanabe et. al. with the following modifications [9]. Cells were detached with Trypsin-EDTA, resuspended in PBS, and then collected by centrifuging at 1200 rpm for 5 min. The cells were further washed with PBS and then concentrated as in the previous step. The concentration of the cells was adjusted to 4.5×10⁶ cells/mL in PBS using a hemocytometer, and 100 μL was aliquoted to specific wells of a 96-well microtiter plate (Maxisorp plates from Nunc). The Maxisorp plates used here have a high protein-binding capacity. Thus, the plates can be used to run assays with either the cells or the phage immobilized in the wells. Next, 50 μL of a 0.15% glutaraldehyde in PBS solution was added to the wells at 4° C., and the solution was gently mixed by pipetting. The ELISA plate was then centrifuged at 1200 rpm for 10 min at 4° C., followed by overnight incubation at 4° C.

Day 2: The cell solution was gently removed, and the wells were blocked with 200 μL/well of blocking buffer containing 100 mM glycine, 1% gelatin and 0.1% w/v BSA (bovine serum albumin) in PBS. The plate was incubated overnight (˜20-22 h) at room temperature.

Separately, phage were prepared before attachment to PEG and PEGylated ligands. Phage (10 nM in 100 μL of PBS) and 1 μL of K₁₄-alkyne (525 μM in water) were thoroughly mixed by pipetting ˜25 times. For phage wrapped with PEGylated ligands, phage were mixed with 0.75 μL of K₁₄-Cys (525 μM in water). For mixed wrapping on the phage surface, 0.5 μL of K₁₄-alkyne was pre-mixed with 0.75 μL of K₁₄-Cys, and then mixed with 100 μL of 10 nM phage. The solution was shaken at room temperature for 15 min on an orbital shaker. Next, 2 μL of PEGylated ligand (625 μM in water) was added to the appropriate wells. For the dual ligand combinations, the PEGylated ligands were pre-mixed in the desired ratio (a 2:1 molar ratio for example), and then 2 μL of the mixture was added to the appropriate wells. The solutions were gently mixed by pipetting, and incubated overnight at 4° C.

Day 3: Next, the click reaction was performed, as previously described, but with the following modifications [1,2,5]. To buffer the pH, triethylammonium acetate was added to a final concentration of 50 mM, followed by the addition of 1.5 μL of 1 mM azide-functionalized PEG. The solutions were mixed by pipetting. Next, ascorbic acid was added to a final concentration of 1 mM and the solutions were mixed by gently pipetting. Then, copper sulfate was added to a final concentration of 1.5 mM, followed by pipetting to mix the solutions. Water was added to the other wells to maintain consistent phage concentrations. The plate was incubated at room temperature for 30 min.

The wells of the ELISA plate were then incubated with the phage samples. The blocking buffer was removed and the wells were gently washed two times with PBS. Next, the phage solution was added to the respective wells and incubated for 45 min. The phage solution was removed, and the wells were washed three times with 300 μL/well of wash buffer PT (0.05% Tween-20 in PBS), once with PBS, and then incubated with horseradish peroxidase-conjugated anti-M13 antibody (100 μL/well, 1:5000 dilution in PBS) for 40 min. The wells were washed three times with PT and once with PBS. The plate was then developed by incubating with HRP substrate solution (100 μL/well; 1 mg/mL o-phenylenediamine dihydrochloride and 0.02% w/v H₂O₂) in citric acid buffer (50 mM citric acid, 50 mM Na₂HPO₄, pH 5.0). The HRP activity was measured spectrophotometrically at 450 nm using a microtiter plate reader (Bio-Tek). The absorbance at 630 nm was subtracted from the absorbance at 450 nm to eliminate background.

Phage-Based Sandwich ELISA for Cell Capture.

To demonstrate cell capture by the PEGylated-ligand phage, the phage were coated on the plate, and cells added before quantifying binding. This assay setup inverts other cell-based phage ELISAs reported here. This experiment is a significant step towards establishing the relevance of this phage architecture for biosensing assays planned in the future. In this assay, the PEGylated phage architecture is immobilized on the plate as demonstrated in FIG. 18. Next, a cell suspension is added to the wells, and the amount of cells captured are then measured spectrophotometrically as detailed here and in the text. The protocol here focuses on experimental details altered from the above-described ELISA; all other conditions remained unchanged.

Day 1: In this phage capture ELISA, specific wells of a 96-well microtiter plate were coated with 100 μL/well of a solution of 10 nM phage pre-wrapped with oligolysine wrappers, as described above. The plate was incubated for 1 h on a shaker at room temperature. The coating solution was removed, and the wells were blocked with 200 μL/well of 0.2% w/v solution of BSA in PBS for 30 min, and washed two times with PT. Next, 98 μL PBS was added per well, followed by PEGylated ligands and incubated overnight at 4° C.

Day 2: Azide-functionalized PEG variants were then conjugated as described above. Separately, the cells were collected and the concentration adjusted as described above; the ELISA plate was then incubated with 100 μL/well of the cell solution or media for 1 h. The wells were washed with PBS and incubated with 100 μL/well of the anti-PSMA antibody, YPSMA antibody (Abcam) at 1:1000 dilution. The wells were then washed with PBS, followed by incubation with horseradish-peroxidase-conjugated anti-mouse antibody (Sigma) at a 1:1000 dilution. The levels of phage binding were quantified as described above.

Conclusions.

In conclusion, this study demonstrates a systematic approach to engineering the phage surface through chemical tailoring. Chemically modifying viruses with PEG addresses a major issue of non-specific adhesion to cellular surfaces, and further engineering allowed specific detection using PEGylated ligands. The reported PEGylated dual ligand combination provides a foundation for applying the phage to cell-based analysis, where highly specific molecular recognition of cells is essential. Optimization of binding affinity required optimization of the PEG length, packing density, point of attachment, linkers and spacers. The versatility of PEG allows such multivariate optimization. This biocompatible polymer is widely available with diverse functionalities for bioconjugation and also has moderately predictable conformations to guide engineering. Furthermore, we demonstrate control over the relative spatial configuration of the ligands using small PEG polymers interdigitated with larger PEG brushes in a general approach applicable to many binding optimization studies. Most importantly, these chemically modified phage could readily distinguish PSMA-positive from PSMA-negative cells, and also identify more aggressive PCa tumor cells. In the future, we will apply such phage to the capture and detection of circulating tumor cells for use in cell-based detectors.

### REFERENCES (MATERIAL AND METHODS)

[1] K. Mohan, K. C. Donavan, J. A. Arter, R. M. Penner and G. A. Weiss, J. Am. Chem. Soc., 2013, 135, 7761-7767; [2] K. Mohan, R. M. Penner and G. A. Weiss, Curr. Protoc. Chem. Biol., 2015, 7, 53-72; [3] R. B. Merrifield, J. Am. Chem. Soc., 1963, 85, 2149-2154; [4] M. Amblard, J.-A. Fehrentz, J. Martinez and G. Subra, Mol. Biotechnol., 2006, 33, 239-254; [5] Lumiprobe http://www.lumiprobe.com/protocols/click-chemistry-dna-labeling (accessed Sep. 7, 2011); [6] V. V Rostovtsev, L. G. Green, V. V Fokin and K. B. Sharpless, Angew. Chem., Int. Ed., 2002, 41, 2596-2599; [7] S. A. Kularatne, K. Wang, H. R. Santhapuram and P. S. Low, Mol. Pharm., 2009, 6, 780-789; [8] C. Tovar, B. Higgins, K. Kolinsky, M. Xia, K. Packman, D. C. Heimbrook and L. T. Vassilev, Mol. Cancer, 2011, 10, 49-59; [9] K. Watanabe, T. Joh, K. Seno, M. Sasaki, I. Todoroki, M. Miyashita, K. Tochikubo and M. Itoh, Clin. Biochem., 2001, 34, 291-295.

What is claimed is:

1. A virial composition comprising:
a) a whole viral particle comprising a charged protein coat, said charged protein coat comprising a plurality of charged coat proteins;
b) a first polymer electrostatically bound to said plurality of charged coat proteins; and
c) a covalent linker linking said first polymer to a recognition moiety.

2. The virial composition of claim 1, wherein said covalent linker is -L¹-L²-L³-L⁴-L⁵-L⁶-, wherein L¹, L², L³, L⁴, L⁵ and L⁶ are independently a bond, —O—, —C(O)O—, —C(O)—, —C(O)NH—, —NH—, —S—, —S(O)₂NH—, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl.

3. The virial composition of claim 2, wherein
L¹ is substituted or unsubstituted heteroalkyl;
L² is substituted or unsubstituted heteroaryl;
L³ is substituted or unsubstituted heteroalkyl;
L⁴ is substituted or unsubstituted heterocycloalkyl;
L⁵ is a substituted or unsubstituted heteroalkyl; and
L⁶ is a bond.

4. The virial composition of claim 3, L⁴ is: 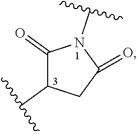 [SMILES: *C1CC(=O)N(*)C1=O]
wherein the carbon at the 3 position is covalently attached to L⁵.

5. The virial composition of claim 4, wherein L⁵ is —S—CH₂—CH(NH₂)—C(O)— or —S—CH₂—CH(C(O)OH)—NH—, wherein the sulfur of L⁵ is attached to L⁴.

6. The virial composition of claim 2, wherein L³ comprises a polyethylene glycol linker.

7. The virial composition of claim 6, wherein said polyethylene glycol linker comprises 2 to 150 oxyethylene units.

8. The virial composition of claim 1, wherein said phage is M13 filamentous phage.

9. The virial composition of claim 1, wherein said first polymer comprises a polypeptide.

10. The virial composition of claim 9, wherein said polypeptide has a net positive charge.

11. The virial composition of claim 9, wherein said polypeptide comprises a polymer of lysine.

12. The virial composition of claim 11, wherein said polymer of lysine is K₂, K₃, K₄, K₅, K₆, K₇, K₈, K₉, K₁₀, K₁₁, K₁₂, K₁₃, K₁₄, K₁₅, K₁₆, K₁₇, K₁₈, K₁₉, or K₂₀.

13. The virial composition of claim 11, wherein said polymer of lysine is K₁₄.

14. The virial composition of claim 1, wherein said first polymer is a net positively charged polymer.

15. The virial composition of claim 1, wherein said first polymer is a net negatively charged polymer.

16. The virial composition of claim 1, wherein said recognition moiety is a cell surface marker binding moiety.

17. The virial composition of claim 16, wherein said cell is a cancer cell.

18. The virial composition of claim 1, wherein said recognition moiety is a polypeptide.

19. The virial composition of claim 18, wherein said polypeptide is an antibody or a fragment thereof.

20. A complex comprising a virial composition of claim 1 and a cell, wherein said recognition moiety of said virial composition is bound to said cell.

21. The complex of claim 20, wherein said cell is a cancer cell.

22. The complex of claim 21, wherein said cancer cell comprises a tumor cell antigen to which the recognition moiety of said virial composition binds.

23. A pharmaceutical composition comprising a virial composition of claim 1 and a pharmaceutically acceptable carrier, diluent or excipient.

24. A method for detecting a cancer cell in a subject, said method comprising:
a) contacting a biological sample of said subject with one or more virial compositions of claim 1, wherein the recognition moiety of one or more virial compositions is a cancer cell surface marker binding moiety, and
b) detecting a cell-virial composition complex, wherein presence of said complex indicates presence of a cancer cell in said subject.

25. The method of claim 24, wherein said virial composition is immobilized to a solid support.

26. The method of claim 24, wherein said one or more virial compositions all comprise the same recognition moiety.

27. The method of claim 24, wherein said virial compositions comprise different recognition moieties.

28. The method of claim 24, wherein said detecting comprises an antibody based reaction.
